SEQUENCE LISTING (25 sequences)

SEQ ID NO: 1 - DNA, 8137 bp, Fusarium fujikuroi IMI 58289
atggtagtaatcgaatcacccccgagtaccgctgtaggctctgccgaagagatacctcaagccatccacaagcatgaggggtctgctttgccggttgctatagtgggcatgggtatgagactgcccggtggaatccatacctgtgatgacctttggaaaatgcttttagggaagcgttcgacccggtgtgagattccatcaactagattctcggttgacggattccacagcccaaccagcaagcctggatctattgccatgcgtcacggccacttcctggacgaccaagatgacctgcaacggttggacacctctttcttctccatggggatgacagaagtgagtgatatagatccacagcagaggatgctcctggaagtggcctatgagtgtatgcaatcttcgggccaaaccgattggcgggggagcaatataggctgttacgttggtgtatggggagaggtatgaatggccgttgacttttacccaacgatctatagctaatctgagtataggattggctggatcttcacagcaaagatttatacgattccggcacatatcgcgtctccggtgggcatgactttgccatttccaaccgtattagttacgaatatgacctcaaaggcccctccttcactatcaaagccggctgctcgtcatccctgatagctcttcatgaagcagtaagggccattcgggcaggagactgtgatggtgctattgtggccggcaccaacctgattttctcaccgaccatgtctatggctatgacagagcaaggcgttttgtccccagatgctatgtgcaaaacctttgatgagaaagcaaacggctatgcaagaggcgaggcaatcaatgccatcttcttgaaacctttagacgatgccctccgggacggagatcccatcagggctgtagtcagggcaacatcttcaaattcggatggcaaaactcccggtatgtcgatgccaagctccgagagccatgaggctttgatccggagggcgtaccaggaggtttgcttggaccccaaggacacctgcttcgtcgaagcacatggaaccggtacatccgtgggcgatccgctcgaggccacggcaatcgcccgtgtctttggcgggtcatcagaccataagctctacatcgggagtgtcaaacctaaccttggacatagcgagggtgcttcaggcatttcgtcagttatgaaggccattctggctctagaaaaccataccatccctccaaacatcaacttcttcacgccgaatcccaaaatccccttttcgggtatgaacatggctgttccagttgatgcaattccctggcctgaagaccgagcacgtcgggtcagcgtcaactcatttggaataggaggtgcaaatgcccactgcatcatagaaactctcgaagagtacctcggtgcatcgttcacaaagcgacatctggttcctccacttcagaatggaaacggctctgtgggtgcccatgtcagcagtgccgtcgacagcatttcagcgatgaaaatggatttgcacagtaagatgcgcaagagtgcaacagcgcaagtcgcaagtgatagttctaccacagacaacgcttatgcccgcccttcaatggctctctatgttctttccgctgcaaatccgacttcgcttaggcagagtgtgacggactatcaagagtatctgggaagtcataagaccgacccggctgatgtttcatataccctgtgcaaccggagagagcatttgtcgcatcgaacctacggagtggtcactacagaatccgtgaatggcgcacacctcgtccctgatttctctcccctatccaagacaaacaacaacacccctcctgatataaacatgatcttcactggccagggtgcccaatgggctggcatgggaaaggagctcatggacgagtacgagaccttttacaacaccattgcttccttgaatctcgttctatccgaactggagcatcctccggcttgggatcttattgaagaattatccaggtctgcggaatcaagcaatgttggccgtgccgagttttctcagccacttgtatgcgcagttcaagtggccctggttgatctcttgcgatcttggggggttgtcccctctgctgtagtaggccactctagtggcgagatggctgctgcctacgcagctggggctgtatcctcccacgaggccatcaaaatcgcctactatcgtggctatgtcaatcaacaatacaagcgagatggcggcatggctgctgttggaatgggagcccaagaagttactccttacctcgtcgacggtgtaactgttgcctgtgagaacagtccccagagtgttacactctcgggagacaaaggtgcacttggggaggtctgccagaagatcacggagcaatttccagattgctttattcgccagctcaaggtcaacgtcgcataccactctcgtaagtgcaacaaccggatatccttcaatgaagcctttactgacaacccaacgccgtctagatcacatgcaagacattggtccagtctttgaggctctactgaaagacaaagtctatagccaagctccagccatacctttcttctccagtgtttctgtcgcaagaattactgaaccaaggagactcgatgctacctattggcgccaaaatctggagagccctgtacgtttcgctggtgctatgaagcttttgctcgaggctcgagagaacgtagcgtcaaagcaggtgtttgtcgaaattgggcctcacagcgcattggctggaccactccgtcaaatcttcatgactcatggcaaaggaaaggaagcctatgcctcagccatgattcgtggccaaaactgcaccgagtcagtcctcagacttgctggcgagctattctgccacggatcatcactgcaattgtcaaatgtgacggccgagggtgatgttgtcgttgacttgccaccttacccttggaaccatgacaaggagtattggtctgagagccgagtcagcagggactggagattccggaaattccccaaccacgaacttcttggctcgcgaacacttgagagcagcaatctacaccctgagtggcgtaatgtgatcaggcttgacaaggtccactggctacgagatcaccaggtcctcaatgatgtcgtctttccttgcgcagggtacctagcgatggcggttgaggcggtgcgacaggtagccggatcgcctgagacagaaggtttcactttaaagagcgttgtcgtccaatctcctctagttatcactgacacgaaaccggttgagatgctcacaagtctgagacccgtcaggctaaccaacactctcgactcggcttggtgggagttctgcatcgtggcacacaatggcaccagctggatcaagcactgtgaaggacaggtcaggcgaggccaagatgctcaccagaaactggcggccctcctgcaaaacaaatcagttgggaaatactatccccgtctcgtggacaatctctatcccgagcttctgagaatcggcttacgatatgggccatctttccgtggccttgatactgtctcctgtgtacccaatggccggaaggccgccgcgacgttgcatgagacaacagtatcggagtcgtcctacgcgatacatcccaccacaattgaccactgcctccaactcttcttccctgctagctgtgacggaatcttttaccgagccgagaaactatgcgttccaacagaaatcggacgcttgtatcttgccgatggaaagctgtgggaggtagagggtgcccgagctgaagcattggcagcgacaaactccgggggctcgatttctggggccgctatcgtggtgtcccgacatgactctgtcctgctctctctggaggatggaaaattctccccactggaaatggatcttggaggggaaggaaacgcagatttggttggtgcggcacgtcttgaatggaaaccaaacttggactttgccgatatgcatagccttgttcgcccaagtcatggatctatgcatgatggacccgaccttgatctggtagagcgacttacgctgcttgctattctagagatccacgagcgcattgaaggtatcgtcactcctgaggatcacgccgatcagcacatactcagcttccgtacctggattgcagaccaagtctcggcagcagccgagggcaaatatcttggggtggtggcagatgctagagagatcgcatcactccaacgcgaagctcgagcatgcttaatgaccaagctccgccaacaggttttggagactggagctgcgagcgctgcagtcttgattggtcgagttgctgaccactgcgaggaaattgtaaaggggaagcttgagggcatcgaacttctccaagcagacggtggccttactaattattataattatgtcgagtcgcggacggactctgtcgacttcttcgccactgctggtcacactcggcccacgcttcgtgttctcgagattggtgctggcactggaggcggagctcaagtcatccttgaaggactaacaaatggcagagaacgcctgtactcgacatatgcatataccgacatatccgccggattcttcgtcacggctcaggaacgattcaaggcataccaaggccttgatttcagagtcctggatataaccaaggaccccactgaacaaggattcgaggttgaatccttcgatcttatcattgccggcaacgttattcatgccaccccatacttgaaggagactcttgcgaatgtgcggaaactgctggctcccgggggttacttttttcttcaagagctttcacccaaaatgagaatggtaaacttgtgagttgacttccggttcaaacatcgtttggccgagttgcagtaactaaattagtacctctctaggatcatgggcatcttgcctggatggtggcttggagccgcagaaggccgagacaaagagccgtacttagctccgtctcagtgggacatagctcttcaggcggccgggttctcaggcgttgacgctgccatttatgatgctccttacccgtaccacctcaatgcaaacatcatcacgaggccggccaaagactcgacagggcagccgcgagcaacgaaaggacgactaacgctacttcatcgccccgacgatattgaatcttcgagtatcaagcagctgcgtaatgtactcgagtctcgtggtcttgagacaaaaatggtggcattgcaggaacacgaacagctcaaagcagagaaccaagacgtcataatttctctgcttgaactcgtgacgccattcttcagctctatctcagtcgcacagctggaatccttccaaagagtagttgctggactcggcagtacagagatgatctgggtcacacggcctgcacagtatggtataggcgcctcagacgaccctggcttcggtctttcgctgggtttggcacgcacactacgctccgagcagtcgttagccatcacgacgcttgagattggccaagtcaatgatgcatcactcaaagctgtggtcagtctcgtgatgagcattctacatcatcaggaagatggtgctgaatcaacacgcggcgccactaacctggaccccgaccgcgagtacgttgtcgataatggcgttatcaaggtcgctaggtaccacccagtagccttgacacaggatcttgcatctagaggagcgaagccagaggctgtgacgcttcagattggccggatgggactgttacagacgctcggttggatgccgtttccaacgtctgatccaggggatggcgagatcacgattgaacctcgatgcgcaggattaaactttcgagtaagtcctttgacattacctgagactaattcagtatgatgctcacaatggttgataaggatgtacttctctgcatgggcgttgtcgaagcaactggtgttggcattgggcttgagggatctggagttattacaaaggtaggggcaagtgttagcgggcttcaacccggagatcgtgtcttctaccttgccgacaactgcttctccacgcagttgactatttcggctcaacgttgtgccaagattccgtcgcaattggcgttcgaggatgccgctacgatgccatgtgtctatgcgacagttattcactctctgctagacgttggcggactacgaccaggacaatctattctgatccactccgcctgtggtggaattggcatcgcagctctgaacttgtgtcgtcacatcgaaggcctagaggtgagttcgaagcggtgacgcgctgacctacctgtcttgcaatgtgctaactcgactggtgatagatatatactacagtcggaaatgaagaaaaggtacagtatctcgtcgacaattttgggcttccgcgatcaaggatcttcaactcccgggacgcctcgtttctgtacgatatacgagcagccacagagggtcgcggtgttgacgtcgtcctcaacagcctgtctggagaactcctacacgcttcatggcaatgcgtagcaccatatgggaaaatgcttgagattggaaaaagagactttatcggcaaagcacgtctggaaatggatctattcgaggctaacagaagtttcattggtattgacctggctaggtttgactcagccagatgtcagaagtgagtctttttccttcctttctttcctccactacacttgttctcacttctcacttttaccatcaaatattgaaagctgacattgcctccaaggctgctcgaacgcaccgcagcaatggtcgaggccaacagcatacagccaatcaagcccgtcaaagtgttcgacgcatccgatgccgaagaagcctttcgatacatgcagaagggagttcatctggggaagatagtggtgtccattccgtctcagagtagtacgctcctacctgtatctccaaggcctcttcaagtgaagctcaatgcagaagcgagttacctcctcgtgggtggactcggtggactcgggcgtgctgctgcgacatggatggtagaaaggggtgcccggtacctcatcttcttctcccggtcggctggtctaagcgtcagagacaagaccttcttccgagaattagcgtcccagggatgtactgctcaggctgtccgaggtgatgtcctgagcctgcctgacgttgaacgttctgtggcttctgccccagcgggaaagcccatcaggggtgttttgcaaatgtccatggtactgcgcgacaagccttttgctgacatgagcttggaagactgggatacggcagtgaagcccaaggtccacggaacctggaacctacatctcgctgcacctaaagatctcgacttcttttttgcaacaggttccatcagcgggtcgtttggtacccccgggcaggctaattatgctgctggtaacacatatctcacagcgctgtttgagcatcgccgtgccctcggtttgccggcgtctgtgctgcagattgggctgatcgaggacattggctatctggcgcaaaacccggaacgtgctgaggcgcttagagcggctggaggctttttcttgcgaactcgacagctgctcgagggtctgaattgggctctgctctcaagcgatccccatcatcccgagtatcagctgacgattggtctgcggagtgacaaatcactatcggacccagccaaccgcgtaatttggaagaaggacagccgagctgccctgtaccacaaccaagagattgccactgacgctggcgtcggtgacgaccagggcctcaacgcaatccgcctcctagttgcctcatgtgaggaagacccagcgatcttacaggaccaggcgacggtcgagctcgtgacacatgaaattgggaagcgcgtttgcatgttcatgttgcggcctgtcgaagagatggacccaacggcaacgctgacgtcgcttggcgttgactcgctggttaccatcgagattcggaactggatcaagcgtacattcgggggcgttgaagttagcaccctggagattctcaactctggcactattgaaggactggcaaggttaacactagacgggctcaagacccggttcgtccttgctggaaggaatgatggtgatgcgtatctggaaatgaaggctcca

SEQ ID NO: 2 - DNA, 8140 bp, Fusarium MK7
atggtagtaatcgaatcacccccgagtaccgctgtaggctctgccgaagagatacctcaagccatccacaagcatgaggggtctgctttgccggttgctatagtgggcatgggtatgagactgcccggtggaatccatacctgtgatgacctttggaaaatgcttttagagaagcgttcgactcggtgtgagattccatcaactagattctcggttgacggattccacagcccaaccagcaagcctggatctattgccatgcgtcacggccacttcttggacgaccaagatgacctgcaccggttggacacctctttcttctccatggggatgacagaagtgagtgatatagatccacagcagaggatgctcctggaagtggcctatgagtgtatgcaatcttcgggccaaaccgattggcgggggagcaatataggctgttacgttggtgtatggggagaggtatgaatgaccgttgacttttacccaacgacctatagctaatctgagtataggattggctggatcttcacagtaaagatttatacgattccggcacatatcgtgtctccggtgggcatgactttgccatttccaaccgtattagttacgaatatgacctcaaaggcccctcattcaccatcaaagccggttgctcgtcatccctgatagctcttcatgaagcagtaagggccattcgggcaggagattgcgatggcgctatcgtggccggcaccaacctgattttctcaccgaccatgtctatggctatgacggagcagggcgttttgtccccagatgctatgtgcaaaacctttgatgaaaaggcaaacggctatgcaagaggcgaggcaatcaatgccatcttcttgaaacctttagacgatgccctccgggacggagatcccatcagggctgtagtcagggcaacatcttcaaattcggatggaaaaactcccggtatgtcgatgccaagctccgagagccatgaggctttgatccggagggcgtaccaggaggtttgcttggaccccaaggacacctgcttcgtcgaagcacatggaaccggtacatccgtgggcgatccgctcgaggccacggcaatcgcccgcgtctttggcgggtcatcagaccataagctctacatcgggagcgtcaaacctaaccttggacatagcgagggtgcttcaggcatttcgtcagttatgaaggccattttggctctagaaaaccaaacaatccctccaaacatcaacttctccacgccgaatcccaaaatccccttttcggatatgaacatggctgttccagttgatgcaattccctggcctgaagaccgagcacgtcgggtcagcgtcaactcatttggaataggaggtgcaaatgcccactgcatcatagaaactctcgaagagtacctcggtgcatcgctcagaaagcggcatccggttcctccacttcagaatggaaacgacactgtgggcacccatgtcagcagtgccgtcgacagcatttcagcgatgaaaatggacttgcgcagtaagatgcgtaatagtgcggcaactcaagtcgcaagtgatagtttcagcacagaaaacgcctatgcccgcccttcaatggctctctttgtcctttctgctgcaaatccgacatcgcttaggcagagtgtgaccgactatcaagggtatctgaaacgtcataagcccgacccggttgatgtttcatataccctgtgcaaccggagagagcatttgtcgcatcgaacctacggcgtggttaccacagaatctgtgaatgacaccgacctcgtccctgatttctcccctctgtccaagacaaacaacaagacccctcctgatataaatctgatcttcactggccagggtgcccaatgggctggcatgggaaaggagctcatggacgaatacgagaccttctacaacacgattgcttcgttgaatctcgttctatccgaactggaccatcctccaacttgggatctcattgaagaactatccaggcctgcagattcaagcaatgttggccgcgccgaattctcccagccacttgtatgcgcaattcaagtggccttggttgatctgttgcgatcttggggggttgtcccttctgctgtagtaggccactctagtggtgagatggctgctgcatacgcagctggggccatatcctcccaagaggccatcaaaatcgcttactatcgtggctatgtcaatcaacagtacaagcgagatggcggcatggctgctgttggactgggagcccaagaagttactccttaccttgtcgacggtgtcactattgcctgtgagaacagtccccagagcgttacactctcgggagacaagggtgcacttggggaggtctgccacaagatcggggagcaattttcagattgctttattcgccagctcaaggtcaacgtcgcataccactctcgtaagtgcaacagccggatatcctccaatcaagcttttactgacaacccaatgccgcctagatcacatgcaagacattggtccagtctttgaggctctactgaaagacaaagtctatagccaagctccagccatacctttcttctccagtgtttctgtcgcaagaattactgaaccaaggagactcgatgctacctattggcgccaaaatctggagagccctgtacgcttcgctggtgctatgaagcttttgctcgaggctcgagagaacgtagcgtcaaagcaggtgtttgtcgaaattgggcctcacagcgcattggctggaccactccgtcaaatcttcatgagtcatggcaaaggaaaggaagcatatgcctcagccatgattcgtggccagaactgcaccgagtcagtcctcagacttgctggcgagctattttgccacggatcatcactgcaattgtcaaatgtgacggccgagggtgatgttgtcgttgacttgccaccttacccttggaaccatgacaaggagtattggtctgagagccgagtcagcagggactggagattccggaaattccccaaccacgaacttcttggctcgcgaacgctcgagagcagcaatctacaccctgagtggcgtaatgtggtcaggcttgacaaggtccagtggctacgagatcaccaggtcctcaatgatgtcatctttccttgcgcagggtacctagcgatggcggttgaagcggtgcgacaggtagccggatcgtctgagacagaaggtttcactttgaagagcgttgtcgtccaatctgctctagttatcactgatgcgaaaccggttgagatgctcacaagtctgagacccgtcaggctaaccaacactctcgactcggcttggtgggagttctgcatcgtggcacacaatggcaccagctggatcaagcactgtgaaggacaggtcaggcaaggccaagatgctcaccagaaactggccgccctcctgcaaaacaaatcagtcgggaaatactacccccgtctcgtggacaatctctatcccgagcttcagagaatcggcttacgatatgggccatctttccgtggccttgatactgtctcctgtgtacccaatggccagaaggccgccgcgacgttgcttgagacaacagtatcggagtcgtcctacgcgatacatcccaccacaattgaccactgcctccaactcttcttccctgctagctgtgacggaatcttttaccgagccgagaaactatgcgttccaacagcaatcggacgcttgtatcttgcggatggaaagctgtgggaggtagagggtgcccgagctgaagcattggccgcgacaaactccgggggctcgatttctggggccgctatcgttgtgtcccaacatgactctgtcctgctctctctggaggatggtaaattctccccactggaaatggatcttggaggggaaggaaacgcagatttggttggtgcggcacgtctcgaatggaaaccaaacttggactttgccgatatgcatagccttgttcgcccaagtcgtggatctatgcatgatggacccgagcttgatctggtagagcgacttacgctgcttgctattctagagatccacgagcgcattgatggtattgtcactcctgaggatcacgccaatcagcacatactcagctttcgtacctggattgcagaccaagtctcggcagcagtcgagggcaaatatcttggggtggtggcggatgctagagagatcgcatcactccaacgcgaagctcgagcatccttaatgaccaagctccgtcaacaggttttggacactggagctgcgagcgctgcagtcttgattggtcgagttgctgaccactgcgaggaaattgtaaaggggaagcttgagggcatcgaacttctccaagcagaagatggccttactaattattacaattatgtcgagtcgcggacggactctgtcgacttcttcgccactgctggtcacactcggcccacgcttcgtgttctcgagattggtgctggcactggaggcggagctcaagtcatacttgaaggactaacaaatggcagagaacgcctgtactcgacatatgcatataccgacatatccgccggattcttcgtcgcggctcaggaacgattcaaggcataccaaggccttgatttcagagtcctggatataaccaaggaccccactgaacaaggattcgaggctgaatccttcgatcttatcattgccggcaacgttattcatgccaccccatacttgaatgagactcttgcgaatgtgcggaaactactggctcctgggggttactttttccttcaagagctttcacccaaaatgagaatggtaaacttgtgagttgagttccggttcaaacctcgtttggccgagttgcagtaactaaattagtacctctctaggatcatgggcatcttgcctggatggtggcttggagccgcagaaggccgagacaaagagccgtacttagctccgcctcagtgggacatagctctcaaggcggccgggttctcaggcgttgacgctgccatttatgatgctccttacccgtaccacctcaatgcaaacatcatcacgaggccggccaaagactcgacagggcagccacgagcaacgaaaggacgactaacgctgcttcatcgccctggcgatgttgactcttcgagtatcaagcagttgcgtaatgttcttgagtctcgtggtcttgagacaaaaatggtggcattgcaggaacacgaacagctcaaagcagagaaccaagacgtcataatttctctgcttgaactcgtgacgccattcttcagctctatctcagtcgcccagctggaatccttccaaagagtagttgctggactcggcagtacagagatgatctgggtcacacggcctgcacagcatggtataggcgcctcagacgaccctggcttcggtctttcgctgggtctggcacgcacactacgctccgagcagtcgttagccatcacgacgcttgagattggccaagtcaatgatgcatcactcaaagctgtggccagtctcgtgatgagcattctacatcatcaagaagatggtgctgaatcaacacgcggcgccactaacctggacccggaccgcgagtacgttgtcgataatggcgttatcaaggtcgctaggtaccacccagtagccttaacacaggagcttgcatctagagcagcgaagccagaggccgtgacgcttcagattggccggatgggactgttacagacgctcggttggatgccgtttccaacgtctgacccagggtacggcgagatcacgattgaacctcgatgcgcaggattaaattttcgagtaagtcctttcacattacctgaaactaactcattatgatgctgacaatggttgataaggatgtacttctctgcatgggcgttgtcgaagcaactggtgttggcattgggcttgagggatctggagttgtcacaaaggtaggggcaagtgttagcgggcttcaacctggagatcgtgtcttctaccttgccgacaactgcttctccacgcagttgactatttcggctcaacgttgtgccaagattccgtcgaaattggcgtttgaggatgccgctacgatgccatgtgtctatgcgacagttattcactctctgctcgacgttggcggactacgaccaggacaatctattctgatccactccgcctgtggtggaattggcattgcagctctgaacttgtgtcgtaacatcgaaggcctagaggtgagttcgaagcgctgtcgcgctgacctacctgtcttgcaatgtgctaactcgactggtgatagatatatactacagtcggaaatgaagagaaggtacagtatctcgtcgacaattttgggcttccgcgatcaaggatcttcaactcccgggacgcctcgtttctgtacgatatacgagcagccacagagggtcgcggtgttgacctcgtcctcaacagcctgtctggagaactgctgcacgcttcatggcaatgcgtagcaccatatgggaaaatgcttgagattggaaaaagagactttatcggcaaagcacgtctggaaatggatctattcgaggctaacagaagtttcattggtattgacctggctaggtttgactcagccagatgtcagaagtgagtcttttcccttcctttctttcttccactaaacttgttctcacttgtcacttttaccatcaaatattgaaagctgacattgcctccaagactgctcgaacgcaccgcagcaatggtcgaggccaacagcatacagccaatcaagcctgtcaaagtgttcgacgcatccgatgccgaagaagcctttcgatacatgcagaagggagttcatctgggcaagatagtggtgtccatgccgcctcagagtagtacgctcctacctgtatctccaaggcctcttcaagtgaatctcaatgcagaagcgagttacctcctcgtgggtggactcggtggactcgggcgtgctgctgcgacatggatggtagaaaggggggcccggtacctcatcttcttctcccggtcggctggtctaagcgtcagagacaagacattcttccgagaattagcgtcccagggctgtactgctcaggctgtccgaggtgatgtcctgagcctgcctgacgtggaacgttcggtggcttctgccccagcgggaaagcccatcaggggtgttttgcaaatgtccatggtcctgcgcgacaagccttttgctgacatgagcttggaagactgggatacggcagtgaagcccaaggtccacggaacctggaatctacatctcgctgcacccaaagatctcgacttcttctttgcaacaggttccatcagcgggtcatttggtacccccgggcaggctaattatgctgctggtaacacatatctcacagcgctgtttgagcatcgccgtgccctcggtttgccggcgtctgtgctgcagattgggctgatcgaggacattggctacctggcgcaaaacccggaacgtgctgaggcgcttagagcggctggaggctttttcttgcgaactcgacagttgctcgagggtctgaattgggctctgctctcaagcgatccccaccatcccgagtatcagctgacgattggtctgcggagtgacaaatcactatcggacccagccaaccgcgtaatttggaagaaggacagccgagctgccctgtaccacaaccaagagattgccactgacgctggcgccggtgacgaccagagcctcaacgcaatccgcctcctagttgcctcatgtgaggaagacccagcgatcttacaggaccaggcgacggtcgagctcgtgacacatgaaattgggaagcgcgtttgcatgttcatgttgcgacctgtcgaagagatggacccaacagcaacgctgacgtcgcttggcgttgactcgctggttaccatcgagattcggaactggatcaagcgtacattcgggggcgttgaagttagcaccctggagattctcaactctggcactattgaaggactggcaaggttaacactagacgggctcaagacccggttcgtccttgctggaaggaatgatggtgatgcgtatctggaaatgaaggctccgtag

SEQ ID NO: 3 - DNA, 838 bp, Artificial Sequence
  MK7 Homologous Recombination 5′ UTR
caccaaccgaagcaccacgctcttgcgctacgctatagtgtaatttacccaccggtagccaactcttcaagcctctaatcattcgagtcctcctaaccacctcagtgttccgctaagcgcaaattggcaggtggcaataccggcatgcgcaatcatgtcatgtagtgcatatacccgccgtggggatcttagccatttttcttccgccatatgtgccgtgttttccgagtccgacaccatgatccacggcagacttgctcctaagaactttgctcgtgacacggcaaacgataaaggacaaccgtctccgtgcaggtcaaaatacggcttcggcatctacaaatagaaatccgatgtggtccaaatacgaagaagccgtcatgatagatactggtctgaatcggacatccgaagcctggcgattacgttaataaccacggccccatacctgcgcgttagatcaaaccggattctggttctgtaattagtcaatggggcaaattcccctttgcccgatccgattggttccgtgggtgttgattgtactcagacatcactgtacttgacctgaggattcgtgtactagcattttggcaagcagaagagagcaatggacagtctactcggttcttcatcacggaacatgtcatttgtctaaggggactgtgattattgtgcatccgaatgcggcgtgttctctcggatgggaactccatagcgtctagattaccgaagtatcgatggtggttgtagttcagttatgatacataagcatcataagatcaaaaatatcatccgaccaagccttcgtagctcatccaccgtaaccttatactcg

SEQ ID NO: 4 - DNA, 839 bp, Artificial Sequence
  MK7 Homologous Recombination 3′ UTR
aacactgaacaagataacagattctgaggtactacttaacttgagcctttacatacttaaatacatctcgaaaatgctcctgaattatacgctcgtatgtgcatgtgccctataatgatgattccttactactgagcctctactttcgccaacagactggcccacagtagttgtcccctgtatcactgtattctggcggacacccatatcgaacttgttcaactcgttaaaggcgtcctatatttttcagagcacggcaataataagtttctgagtctgtgccatacaacccgctattacagcggagcctaataattcattcatcacaatgctgtttccaccactgcgcaaccagtgtcttcatttctggcgaaggtaaatagaatgaatacataacatagctacccccagaatgtccattctaactacacataaacttcttctacatatctccccttgtccaaccctgctctccattctacaaccgtctttactgctgtttcccctctgttctgatcagtttccaatagcatcggccctagaacctcttgcacgtcatttgccattttcttacctccgcagacaaggaccttggcgccagccacccagaggcgaacaatttcatctcgacatctttcgagcttctcagtaacgtacctgcagccttgggcacctgtggcagaagggtcacggctataggctcttcgcatttgcacgatgcccatggcttcgtaggcttccatttcaatcgagtacagagagtcaagcccgggtcctcgacaaccgaaaaacaggagggccggcccaagcttcgatttggaatcttcagcagccagcct

SEQ ID NO: 5 - DNA, 1892 bp, Artificial Sequence
  hph casette
gctagtggaggtcaacacatcaatgctattttggtttagtcgtccaggcggatcacaaaatttgtgtcgtttgacaagatggttcatttaggcaactggtcagatcagcccacttgtaagcagtagcggcggcgctcgaagtgtgactcttattagcagacaggaacgaggacattattatcatctgctgcttggtgcacgataacttgtgcgtttgtcaagcaaggtaagtgaacgacccggtcataccttcttaagttcgcccttcctccctttatttcagattcaatctgacttacctattctacccaagcatccaaatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctggagccggtcgcggaggccatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtatcgccgatagtggaaaccgacgccccagcactcgtccgagggcaaaggaatagagtagatgccgaccgatccccactaccgcattaagacctcagcgcggccgcaaatttaaataaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccg

SEQ ID NO: 6 - DNA, 38 bp, Artificial Sequence
  HR-L_Forward
gagctcggtacccggggatccaccaaccgaagcaccac

SEQ ID NO: 7 - DNA, 18 bp, Artificial Sequence
  HR-L_Forward_S
caccaaccgaagcaccac

SEQ ID NO: 8 - DNA, 23 bp, Artificial Sequence
  HR-L_Reverse
cgagtataaggttacggtggatg

SEQ ID NO: 9 - DNA, 41 bp, Artificial Sequence
  HPH_Forward
ccaccgtaaccttatactcggctagtggaggtcaacacatc

SEQ ID NO: 10 - DNA, 21 bp, Artificial Sequence
  HPH_Forward_S
gctagtggaggtcaacacatc

SEQ ID NO: 11 - DNA, 42 bp, Artificial Sequence
  HPH_Reverse
ctgttatcttgttcagtgttcggttctgataacgagtaatcg

SEQ ID NO: 12 - DNA, 22 bp, Artificial Sequence
  HPH_Reverse_S
cggttctgataacgagtaatcg

SEQ ID NO: 13 - DNA, 26 bp, Artificial Sequence
  HR-R_Forward
aacactgaacaagataacagattctg

SEQ ID NO: 14 - DNA, 38 bp, Artificial Sequence
  HR-R_Reverse
caggtcgactctagaggatcaggctggctgctgaagat

SEQ ID NO: 15 - DNA, 18 bp, Artificial Sequence
  HR-R_Reverse_S
aggctggctgctgaagat

SEQ ID NO: 16 - DNA, 25 bp, Artificial Sequence
  FUM5_5′UTR_F0b
catcatggctcacagagaatttcat

SEQ ID NO: 17 - DNA, 19 bp, Artificial Sequence
  FUM5_5′UTR_F0
gcgtttgctggtgatcttg

SEQ ID NO: 18 - DNA, 21 bp, Artificial Sequence
  MK7_fum5_exon_1_F1
atggtagtaatcgaatcaccc

SEQ ID NO: 19 - DNA, 20 bp, Artificial Sequence
  MK7_fum5_exon_1_R1
cgggggtgattcgattacta

SEQ ID NO: 20 - DNA, 22 bp, Artificial Sequence
  FUM5_exon_3_F1
aacgattcaaggcataccaagg

SEQ ID NO: 21 - DNA, 22 bp, Artificial Sequence
  FUM5_exon_3_R1
ccttggtatgccttgaatcgtt

SEQ ID NO: 22 - DNA, 21 bp, Artificial Sequence
  MK7_fum5_exon_7_F1
taacactagacgggctcaaga

SEQ ID NO: 23 - DNA, 19 bp, Artificial Sequence
  FUM6_3′_R0
cttgttcagcttcggccaa

SEQ ID NO: 24 - DNA, 20 bp, Artificial Sequence
  FUM6_3′_R0b
agcgcatgctgaaagacagt

SEQ ID NO: 25 - protein, 2580 aa, Fusarium MK7
MVVIESPPSTAVGSAEEIPQAIHKHEGSALPVAIVGMGMRLPGGIHTCDDLWKMLLEKRSTRCEIPSTRFSVDGFHSPTSKPGSIAMRHGHFLDDQDDLHRLDTSFFSMGMTEVSDIDPQQRMLLEVAYECMQSSGQTDWRGSNIGCYVGVWGEDWLDLHSKDLYDSGTYRVSGGHDFAISNRISYEYDLKGPSFTIKAGCSSSLIALHEAVRAIRAGDCDGAIVAGTNLIFSPTMSMAMTEQGVLSPDAMCKTFDEKANGYARGEAINAIFLKPLDDALRDGDPIRAVVRATSSNSDGKTPGMSMPSSESHEALIRRAYQEVCLDPKDTCFVEAHGTGTSVGDPLEATAIARVFGGSSDHKLYIGSVKPNLGHSEGASGISSVMKAILALENQTIPPNINFSTPNPKIPFSDMNMAVPVDAIPWPEDRARRVSVNSFGIGGANAHCIIETLEEYLGASLRKRHPVPPLQNGNDTVGTHVSSAVDSISAMKMDLRSKMRNSAATQVASDSFSTENAYARPSMALFVLSAANPTSLRQSVTDYQGYLKRHKPDPVDVSYTLCNRREHLSHRTYGVVTTESVNDTDLVPDFSPLSKTNNKTPPDINLIFTGQGAQWAGMGKELMDEYETFYNTIASLNLVLSELDHPPTWDLIEELSRPADSSNVGRAEFSQPLVCAIQVALVDLLRSWGVVPSAVVGHSSGEMAAAYAAGAISSQEAIKIAYYRGYVNQQYKRDGGMAAVGLGAQEVTPYLVDGVTIACENSPQSVTLSGDKGALGEVCHKIGEQFSDCFIRQLKVNVAYHSHHMQDIGPVFEALLKDKVYSQAPAIPFFSSVSVARITEPRRLDATYWRQNLESPVRFAGAMKLLLEARENVASKQVFVEIGPHSALAGPLRQIFMSHGKGKEAYASAMIRGQNCTESVLRLAGELFCHGSSLQLSNVTAEGDVVVDLPPYPWNHDKEYWSESRVSRDWRFRKFPNHELLGSRTLESSNLHPEWRNVVRLDKVQWLRDHQVLNDVIFPCAGYLAMAVEAVRQVAGSSETEGFTLKSVVVQSALVITDAKPVEMLTSLRPVRLTNTLDSAWWEFCIVAHNGTSWIKHCEGQVRQGQDAHQKLAALLQNKSVGKYYPRLVDNLYPELQRIGLRYGPSFRGLDTVSCVPNGQKAAATLLETTVSESSYAIHPTTIDHCLQLFFPASCDGIFYRAEKLCVPTAIGRLYLADGKLWEVEGARAEALAATNSGGSISGAAIVVSQHDSVLLSLEDGKFSPLEMDLGGEGNADLVGAARLEWKPNLDFADMHSLVRPSRGSMHDGPELDLVERLTLLAILEIHERIDGIVTPEDHANQHILSFRTWIADQVSAAVEGKYLGVVADAREIASLQREARASLMTKLRQQVLDTGAASAAVLIGRVADHCEEIVKGKLEGIELLQAEDGLTNYYNYVESRTDSVDFFATAGHTRPTLRVLEIGAGTGGGAQVILEGLTNGRERLYSTYAYTDISAGFFVAAQERFKAYQGLDFRVLDITKDPTEQGFEAESFDLIIAGNVIHATPYLNETLANVRKLLAPGGYFFLQELSPKMRMVNLIMGILPGWWLGAAEGRDKEPYLAPPQWDIALKAAGFSGVDAAIYDAPYPYHLNANIITRPAKDSTGQPRATKGRLTLLHRPGDVDSSSIKQLRNVLESRGLETKMVALQEHEQLKAENQDVIISLLELVTPFFSSISVAQLESFQRVVAGLGSTEMIWVTRPAQHGIGASDDPGFGLSLGLARTLRSEQSLAITTLEIGQVNDASLKAVASLVMSILHHQEDGAESTRGATNLDPDREYVVDNGVIKVARYHPVALTQELASRAAKPEAVTLQIGRMGLLQTLGWMPFPTSDPGYGEITIEPRCAGLNFRDVLLCMGVVEATGVGIGLEGSGVVTKVGASVSGLQPGDRVFYLADNCFSTQLTISAQRCAKIPSKLAFEDAATMPCVYATVIHSLLDVGGLRPGQSILIHSACGGIGIAALNLCRNIEGLEIYTTVGNEEKVQYLVDNFGLPRSRIFNSRDASFLYDIRAATEGRGVDLVLNSLSGELLHASWQCVAPYGKMLEIGKRDFIGKARLEMDLFEANRSFIGIDLARFDSARCQKLLERTAAMVEANSIQPIKPVKVFDASDAEEAFRYMQKGVHLGKIVVSMPPQSSTLLPVSPRPLQVNLNAEASYLLVGGLGGLGRAAATWMVERGARYLIFFSRSAGLSVRDKTFFRELASQGCTAQAVRGDVLSLPDVERSVASAPAGKPIRGVLQMSMVLRDKPFADMSLEDWDTAVKPKVHGTWNLHLAAPKDLDFFFATGSISGSFGTPGQANYAAGNTYLTALFEHRRALGLPASVLQIGLIEDIGYLAQNPERAEALRAAGGFFLRTRQLLEGLNWALLSSDPHHPEYQLTIGLRSDKSLSDPANRVIWKKDSRAALYHNQEIATDAGAGDDQSLNAIRLLVASCEEDPAILQDQATVELVTHEIGKRVCMFMLRPVEEMDPTATLTSLGVDSLVTIEIRNWIKRTFGGVEVSTLEILNSGTIEGLARLTLDGLKTRFVLAGRNDGDAYLEMKAP

(19) United States
(12) Patent Application Publication
(10) Pub. No.: US 2022/0315881 A1
(43) Pub. Date: Oct. 6, 2022

(54) RECOMBINANT FUNGAL STRAINS WITH REDUCED LEVELS OF MYCOTOXINS

(71) Applicant: THE FYNDER GROUP, INC., Chicago, IL (US)

(72) Inventor: Richard E. MACUR, Manhattan, MT (US)

(21) Appl. No.: 17/614,710

(22) PCT Filed: May 29, 2020
(86) PCT No.: PCT/US2020/035122

§ 371 Date: Nov. 29, 2021

Related U.S. Application Data
(60) Provisional application No. 62/854,170, filed on May 29, 2019.

(51) Int. Cl. C12N 1/14; C12N 15/80; C12N 15/90; A23L 31/00

(52) U.S. Cl. CPC C12N 1/14; C12N 1/145; C12N 15/80; C12N 15/902; A23L 31/00; C12R 2001/77

(57) ABSTRACT

Described herein are recombinant edible filamentous fungal strain comprising a genetic modification of a gene in a mycotoxin biosynthesis pathway and methods to produce such recombinant edible filamentous fungal strains. Such recombinant strains comprise reduced levels of mycotoxins. Also described are food materials comprising the recombinant edible filamentous fungal strains described herein, and methods to prepare them.

### CROSS-REFERENCE TO RELATED APPLICATIONS

This application claims the benefit of priority of U.S. Provisional Patent Application 62/854,170, filed 29 May 2019, the entirety of which is incorporated herein by reference.

### FIELD

The invention relates to recombinant edible filamentous fungal strains which produce reduced levels of mycotoxins, methods to prepare them, biomats comprising the recombinant edible filamentous fungal strains and their use in preparing food materials.

### BACKGROUND

Mycotoxins are low-molecular-weight fungal metabolites that are toxic to animals. These metabolites are structurally diverse and differ in their mechanisms of toxicity. They are of concern in food safety and agriculture because of their toxic properties and their frequent accumulation in crops used for food and feed. Examples of mycotoxins include fumonisins, fusarins, fusaric acid, aflatoxins, ochratoxins, zearalenone, etc.

Fumonisins, are produced by a number of fungi including certain species of the genus Fusarium, including F. anthophilum, F. fujikuroi, F. nygamai, F. oxysporum, and F. proliferatum (Munkvold and Desjardins 1997).

Fumonisins are structurally similar to sphinganine, a sphingolipid biosynthetic intermediate. Fumonisins, and other sphinganine-analogue mycotoxins disrupt sphingolipid metabolism in plants and in animals by inhibiting the enzyme sphinganine N-acyltransferase and causing the accumulation of sphingoid bases, which is thought to be responsible for the majority of fumonisin-induced mycotoxicosis.

At least 28 fumonisins have been isolated from fungi, and they are classified into four groups, fumonisin A, B, C, and P series. The A-series is structurally similar to the B-series with the exception of a C2 amino-acylated functionality. The P-series fumonisins have a 3-hydroxypyridinium moiety at the C2 position in contrast to the free amino group found in the B and C-series. The C-series is distinct from the A, B, and P-series in that it has a C19 backbone resulting from the condensation of an acyl chain with glycine as opposed to an alanine residue.

Fumonisins B (FB) are the major fumonisins produced in nature, and are polyketides consisting of a linear 19- or 20-carbon backbone with hydroxyl, methyl, and tricarballylic acid moieties at various positions along the base chain. Examples include fumonisin 1 (FB1), fumonisin B2 (FB2), and fumonisin B3 (FB3) and fumonisin B4 (FB4). Further, FB6 is an isomer of FB1, having hydroxyl groups at C3, C4, and C5, instead of at C3, C5, and C10 in FB1.

Structure of FB1 is shown below. (ApSimon, Environmental Health Perspectives 2001,109:245-49.

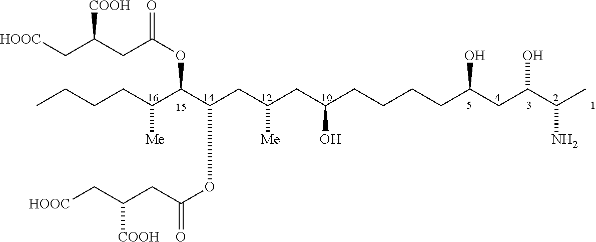 [SMILES: CCCC[C@@H](C)[C@@H](OC(=O)C[C@@H](CC(=O)O)C(=O)O)[C@H](C[C@@H](C)C[C@H](O)CCCC[C@@H](O)C[C@H](O)[C@H](C)N)OC(=O)C[C@@H](CC(=O)O)C(=O)O]

The World Health Organization (WHO) guidelines for human consumption is 2 μg/Kg body weight per day. The current FDA guidelines for human and animal are as follows.

| | | Total Fumonisins |
|---|---|---|
| Human foods | Product | (FB1 + FB2 + FB3) |
| | Degermed dry milled corn products (e.g., | 2 parts per million (ppm) |
| | flaking grits, corn grits, corn meal, corn flour | |
| | with fat content of <2.25%, dry weight basis) | |
| | Whole or partially degermed dry milled corn | 4 ppm |
| | products (e.g., flaking grits, corn grits, corn | |
| | meal, corn flour with fat content of >2.25%, | |
| | dry weight basis) | |
| | Dry milled corn bran | 4 ppm |
| | Cleaned corn intended for masa production | 4 ppm |
| | Cleaned corn intended for popcorn | 3 ppm |
| | Degermed dry milled corn products (e.g., | 2 parts per million (ppm) |
| | flaking grits, corn grits, corn meal, corn flour | |
| | with fat content of <2.25%, =“” dry = “” | |
| | weight = “”> | |
| Animal Feeds | Corn and corn by-products intended for: | |
| | Equids and rabbits | 5 ppm (no more than 20% |
| | | of diet)** |
| | Swine and catfish | 20 ppm (no more than |
| | | 50% of diet)** |
| | Breeding ruminants, breeding poultry and | 30 ppm (no more than |
| | breeding mink* | 50% of diet)** |
| | Ruminants > 3 months old being raised for | 60 ppm (no more than |
| | slaughter and mink being raised for pelt | 50% of diet)** |
| | production | |
| | Poultry being raised for slaughter | 100 ppm (no more than |
| | | 50% of diet)** |
| | All other species or classes of livestock and | 10 ppm (no more than |
| | pet animals | 50% of diet)** |

*Includes lactating dairy cattle and hens laying eggs for human consumption
**Dry weight basis

Fusarins are another class of mycotoxins of the polyketide family, produced mainly by the fungi of the genus Fusarium. Examples include Fusarin A, B, C, D and F. Fusarin C has been reported to induce mutagenesis in mammalian cells and to cause immunosuppression. The structure of Fusarin C is shown below.

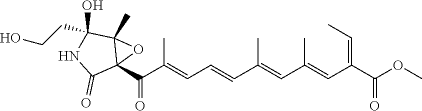 [SMILES: C\C=C(/C=C(C)/C=C(C)/C=C/C=C(\C)C(=O)[C@]12O[C@@]1(C)[C@@](O)(CCO)NC2=O)C(=O)OC]

Fusaric acid is a mycotoxin with low to moderate toxicity. Fusaric acid is thought to augment the overall toxicity of other mycotoxins. Thus, the major importance of fusaric acid to animal toxicity may be synergistic interactions with other naturally co-occurring mycotoxins. Structure of fusaric acid is shown below.

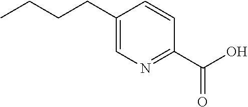

With the increase in global demand for food, in particular nutritious food with high protein content, coupled with a growing awareness of environmental costs associated with the production of animal-based foods, search for innovative alternatives to traditional non-animal sources of food has intensified. In the recent years, edible fungi have emerged as a potential source of a non-animal based food. These include, inter alia, Fusarium venenatum formerly classified as Fusarium graminearum) marketed as Quorn. A novel strain called Fusarium MK7 (deposited as ATCC Deposit No. PTA-1069) has been described in WO/2016/004380. MK7 was found to be a source of high quality nutritious protein-rich food. See e.g., WO/2017/151684, WO 2019/046480 and PCT/US20/20152 describing MK7 and other edible filamentous fungal strains. The disclosures of each of these applications is incorporated herein by reference in their entirety. The presence of mycotoxins can be problematic in edible fungi, and if the level of mycotoxins is above recognized thresholds, it must be reduced for any such food products to be deemed safe for consumption.

Some edible fungi, notably MK7, naturally contain low or negligible (undetectable) levels of mycotoxins. However, with an increasing focus on production of foods comprising edible filamentous fungi, there is a need in the art for edible fungal strains in which production of mycotoxins, such as fumonisins, fusarin and fusaric acid, is further reduced or abolished.

### SUMMARY

In one aspect, the present invention includes a recombinant edible filamentous fungal strain, wherein the recombinant strain comprises a genetic modification of a gene in a mycotoxin biosynthesis pathway and wherein the recombinant strain produces a reduced level of the mycotoxin. In some embodiments, the mycotoxin is a polyketide. Examples of such mycotoxins are fumonisin, fusarin or fusaric acid. In some embodiments, the genetic modification comprises a modification of a gene encoding a polyketide synthase. Examples of a polyketide synthase include Fum1, Fus1 and Fub. Thus, for example, in some embodiments, the modification may be of one or more genes selected from Fum1, Fus1 and Fub1. In some embodiments, the genetic modification comprises a modification in another gene involved in the biosynthesis pathway.

In some embodiments, the mycotoxin may be fumonisin and the genetic modification may be of one or more of genes selected from Fum1, Fum2, Fum3, Fum6, Fum7, Fum8, Fum10, Fum11, Fum13, Fum14, Fum15, Fum16, Fum17, Fum18, Fum19, Fum20 and Fum21. In some embodiments, the genetic modification may be of one or more of genes selected from Fum1, Fum6 and Fum8. In some embodiments, the Fum1 gene may be at least 70%, or at least 80%, or at least 90%, or at least 95%, or at least 96%, or at least 97%, or at least 98%, or at least 99% identical to SEQ ID NO: 2. In some embodiments, Fum1 comprises SEQ ID NO: 2. In some embodiments, the mycotoxin may be fusarin and the genetic modification may be of one or more of genes selected from Fus1, Fus2, Fus3, Fus4, Fus5, Fus6, Fus7, Fus8 and Fus9. In some embodiments, the mycotoxin is fusaric acid and the genetic modification may be of one or more of genes selected from Fub1, Fub2, Fub3, Fub4, Fub5, Fub6, Fub7, Fub8, Fub9, Fub10, Fub11 and Fub 12.

In some embodiments, the genetic modification may comprise a deletion of the gene. The deletion may be partial or complete. In some embodiments, the genetic modification of the gene may be performed using homologous recombination. In some embodiments, the genetic modification of the gene may be performed using CRISPR or TALEN. In some embodiments, the genetic modification of the gene may be performed in combination with introducing a selectable marker, for example hygromycin resistance. In some embodiments, no selectable marker may be used.

In some embodiments, the edible filamentous fungus may be a Fusarium species. In some embodiments, the edible filamentous fungus may be MK7 (ATCC PTA-10698) or Fusarium venenatum. In some embodiments, the recombinant strain may express less than about 10 ppm, or less than about 2 ppm of the mycotoxin (fumonisin, fusarin or fusaric acid) on a dry weight basis. In some embodiments, the recombinant strain does not produce a functional fumonisin, fusarin, fusaric acid or a combination thereof. In some embodiments, the fumonisin comprises fumonisin FB, for example, FB1, FB2 and FB3. In some embodiments, the fusarin comprises fusarin C.

In another aspect, the present invention includes a biomat comprising the recombinant edible filamentous fungal strain. In another aspect, the present invention includes a method of producing the biomat by surface fermentation method. The method may comprise the steps of inoculating an artificial liquid growth medium with an effective amount of the recombinant edible filamentous fungal strain; incubating the inoculated filamentous fungi to produce a filamentous fungal biomat on the surface of the liquid medium; and harvesting the filamentous fungal biomat.

In another aspect, the present invention includes a method to prepare a food material comprising culturing and harvesting the recombinant edible filamentous fungal strains described herein. The method may comprise inoculating an artificial liquid growth medium with an effective amount of a biologically pure culture of the recombinant edible filamentous fungal strain and incubating the inoculated filamentous fungi to produce a filamentous fungal biomat on the surface of the liquid medium. In another aspect, the present invention includes a food material comprising the recombinant edible filamentous fungal strains described herein.

### DESCRIPTION OF DRAWINGS

FIG. 1 shows the biosynthetic pathway for fumonisins.

FIG. 2 shows a gel electrophoresis analysis in which the ˜13 kb band expected from the Fum1 harboring wild type (WT) strain was not present in the Δ8b-Fum1 and Δ9c-Fum1 mutant strains.

FIG. 3 shows arrangement of genes implicated in fumonisin biosynthesis.

FIG. 4 shows arrangement of genes implicated in fusarin C biosynthesis.

FIG. 5 shows arrangement of genes implicated in fusaric acid biosynthesis.

### DETAILED DESCRIPTION

Described herein are edible recombinant filamentous fungal strains which comprise a genetic modification in a gene involved in a mycotoxin biosynthesis pathway. Such recombinant strains contain reduced levels of mycotoxins compared to the unmodified strains and can be suitable for human and/or animal consumption without further processing to lower mycotoxin levels. By eliminating or minimizing the steps typically required for the removal of mycotoxins from edible fungal strains, they provide valuable processing and economic advantages.

The genetic modification of a gene in the mycotoxin biosynthesis pathway results in the recombinant strain producing a reduced level of the mycotoxin compared to the unmodified or naturally occurring strain that does not have the genetic modification and that is grown under substantially the same growth conditions, e.g. same media, temperature, pH, growth period etc. In some embodiments, the reduction in the level of the mycotoxin may be 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%, or any number in the range of 10-100% compared to an unmodified strain that does not have the genetic modification and that is grown under substantially the same growth conditions.

Before the present invention is further described, it is to be understood that this invention is not limited to particular embodiments described, and as such may vary. It is also to be understood that the terminology used herein is for the purpose of describing particular embodiments only and is not intended to be limiting.

It must be noted that as used herein and in the claims, the singular forms “a,” “an,” and “the” include plural referents unless the context clearly dictates otherwise. It is further noted that the claims may be drafted to exclude any optional element. For example, a genetic modification refers to one or more genetic modifications. As such, the terms “a”, “an”, “one or more” and “at least one” can be used interchangeably. Similarly, the terms “comprising”, “including” and “having” can be used interchangeably.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

In one aspect, the present invention comprises a recombinant edible filamentous fungal strain, wherein the recombinant strain comprises a genetic modification of a gene involved in a mycotoxin biosynthesis pathway and wherein the recombinant strain produces reduced levels of the mycotoxin. The gene may encode a polypeptide (such as an enzyme) that is required in mycotoxin biosynthesis, so that the genetic modification of the gene reduces the expression or activity of the polypeptide.

For instance, in some embodiments, the mycotoxin is a polyketide. Examples of polyketide mycotoxins include fumonisins, fusarins and fusaric acid.

In some embodiments, the genetic modification may be in a gene encoding a polyketide synthase. For example, Fum1 encodes a polyketide synthase involved in the biosynthesis of fumonisins. Fus1 encodes a polyketide synthase involved in the biosynthesis of fusarins. Fub1 encodes a polyketide synthase involved in the biosynthesis of fusaric acid. These genes have been isolated from several fungal strains and their sequences determined. For example, the nucleotide sequence of Fum1 (also called Fum5, fumonisin PKS) isolated from Fusarium fujikuroi is available as accession no. IMI58289: HF679031; the nucleotide sequence of Fus1 (also called FusA, fusarin PKS) isolated from Gibberella fujikuroi is available as accession no. IMI58289: JX308619.1; the nucleotide sequence of Fub1 (fusaric acid PKS) isolated from Fusarium fujikuroi is available as accession no. IMI58289: XM_023573799.

Thus, in some embodiments, the recombinant strain comprises a modification of the Fum1 gene and produces reduced levels of fumonisins. In some embodiments, the recombinant strain comprises a modification of the Fus1 gene and produces reduced levels of fusarin C. In some embodiments, the recombinant strain comprises a modification of the Fub1 gene and produces reduced levels of fusaric acid.

In some embodiments, the recombinant strain may comprise a modification of one or more of Fum1, Fus1, and Fub1 genes. For example, in some embodiments, it may comprise a modification of Fum1 and Fus1 genes, and produce reduced levels of fumonisins and fusarin C. In some embodiments, it may comprise a modification of Fum1 and Fub1 genes, and produce reduced levels of fumonisins and fusaric acid. In some embodiments, it may comprise a modification of Fus1 and Fub1 genes, and produce reduced levels of fusarin C and fusaric acid. In some embodiments, it may comprise a modification of Fum1, Fus1 and Fub1 genes, and produce reduced levels of fumonisins, fusarin C and fusaric acid.

In some embodiments, the recombinant strain comprises a genetic modification of a gene in the fumonisin biosynthesis pathway and produces reduced levels of fumonisin.

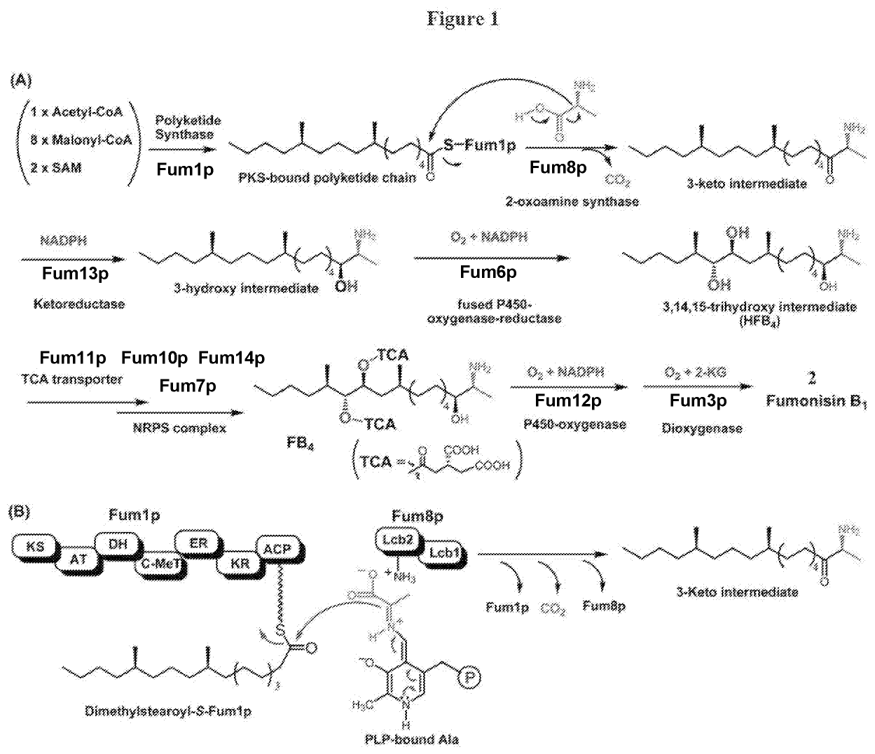

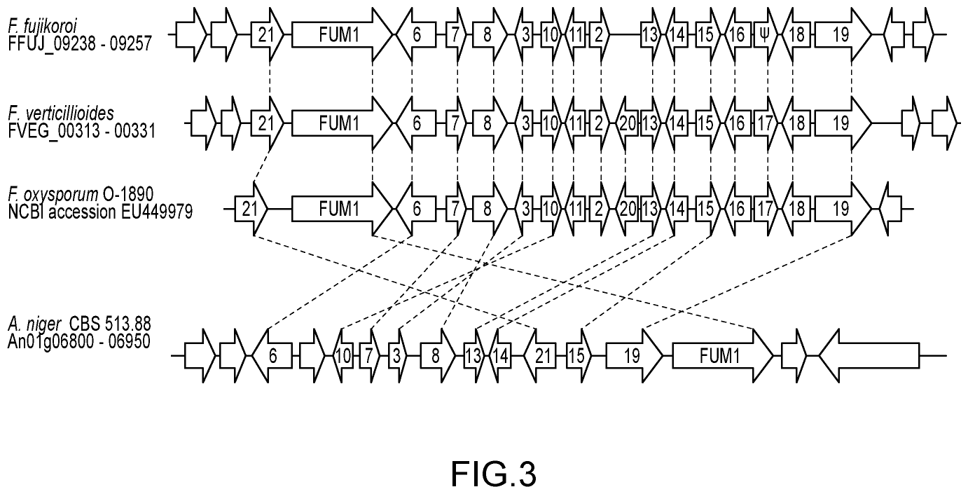

The fumonisin biosynthetic gene cluster involved in the fumonisin biosynthesis pathway was previously identified from the filamentous fungal strain Fusarium verticillioides. See e.g. Huffman et al., 2010; Visentin et al., 2011; Medina et al.; 2013, Rocha et al., 2015. A schematic presentation of fumonisin biosynthesis mechanism is shown in FIG. 1 (Huffman et al., 2010). These genes have been isolated from several fungal strains and their sequences determined and known in the art. The fumonisin biosynthetic gene cluster comprises 16 or 17 transcriptionally coregulated genes, which include genes designated as Fum1, Fum2, Fum3, Fum6, Fum7, Fum8, Fum10, Fum11, Fum13, Fum14, Fum15, Fum16, Fum17, Fum18, Fum19, Fum20 and Fum21. (Fum1 was later found to be the same as Fum5. Fum2 and Fum3 were later found to be the same as Fum12 and Fum9, respectively. Visentin et al. 2011.) Studies have demonstrated that these genes are involved in fumonisin production. FIG. 3 shows the gene arrangement and comparisons between four fumonisin producing filamentous fungi, namely Fusarium fujikuroi, Fusarium verticillioides, Fusarium oxysporum O-1890 and Aspergillus niger CBS 513,88 (Weimann et al., 2013). Although analytical testing indicated that a negligible amount of fumonisin was produced by MK7, analysis of its DNA sequence revealed that MK7 genome possesses a complete set of 16 genes for fumonisin biosynthesis as in Fusarium fujikuroi.

The gene Fum1 encodes a polyketide synthase, which plays a key role in the biosynthesis, as it catalyzes the synthesis of a linear polyketide that forms the backbone structure of fumonisins. Fum8 encodes an alpha-aminotransferase, which is involved in adding amine groups to the backbone. The remaining genes encode proteins that take part in further processing of the backbone. Fum13 encodes a C-3 carbonyl reductase. Fum2, Fum3, Fum6 and Fum15 encode cytochrome P450 oxygenases that catalyse oxygenation. Fum7, Fum 10, Fum11, Fum14 and Fum16 are required for tricarballylic acid esterification. Fum17 and Fum 18 are longevity assurance factors; and Fum19 encodes a protein highly similar to ABC multidrug transporters, which can possibly reduce cellular concentration of toxins. These have been suggested as being involved in fumonisin self-protection and sphingolipid metabolism.

In one embodiment, the recombinant strain comprises a genetic modification of any one or more of the genes involved in the fumonisin biosynthesis pathway. Thus, the recombinant strain may comprise a genetic modification of any one or more of the genes selected from the group Fum1, Fum2, Fum3, Fum6, Fum7, Fum8, Fum 10, Fum11, Fum 13, Fum14, Fum 15, Fum16, Fum17, Fum 18, Fum19, Fum20 and Fum21. In some embodiments, the recombinant strain may comprise a genetic modification of the Fum1 gene. In some embodiments, the recombinant strain may comprise a genetic modification of the Fum6 gene. In some embodiments, the recombinant strain may comprise a genetic modification of the Fum8 gene.

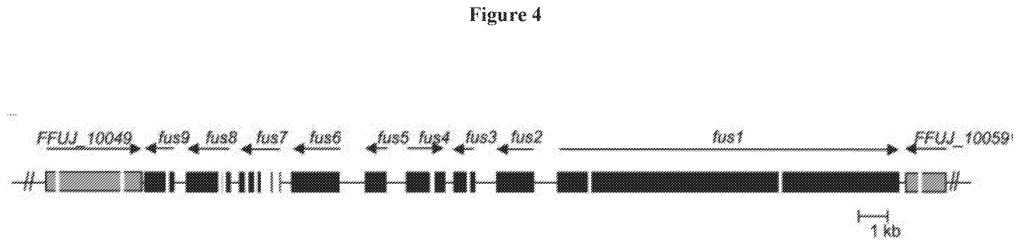

In some embodiments, the recombinant strain comprises a genetic modification of a gene in the fusarin biosynthesis pathway and produces reduced levels of fusarin. Biosynthesis of fusarin C is linked to a cluster of 9 genes, which include Fus1, Fus2, Fus3, Fus4, Fus5, Fus6, Fus7, Fus8 and Fus9. These genes encode a polyketide synthase (Fus1), cytochrome p450 (Fus8), a hydrolase (Fus2) and a methyltransferase (Fus9). FIG. 4 shows the gene arrangement in Fusarium fujikuroi (Niehaus et al., 2013). These genes have been isolated from several fungal strains and their sequences determined and known in the art. Although no fusarin C was detected in MK7 in analytical testing, surprisingly analysis of its DNA sequence revealed that MK7 genome possesses a complete set of 9 genes for fusarin C biosynthesis as in Fusarium fujikuroi. Thus, there is a possibility that MK7 may have a potential to produce fusarin C under some culture conditions.

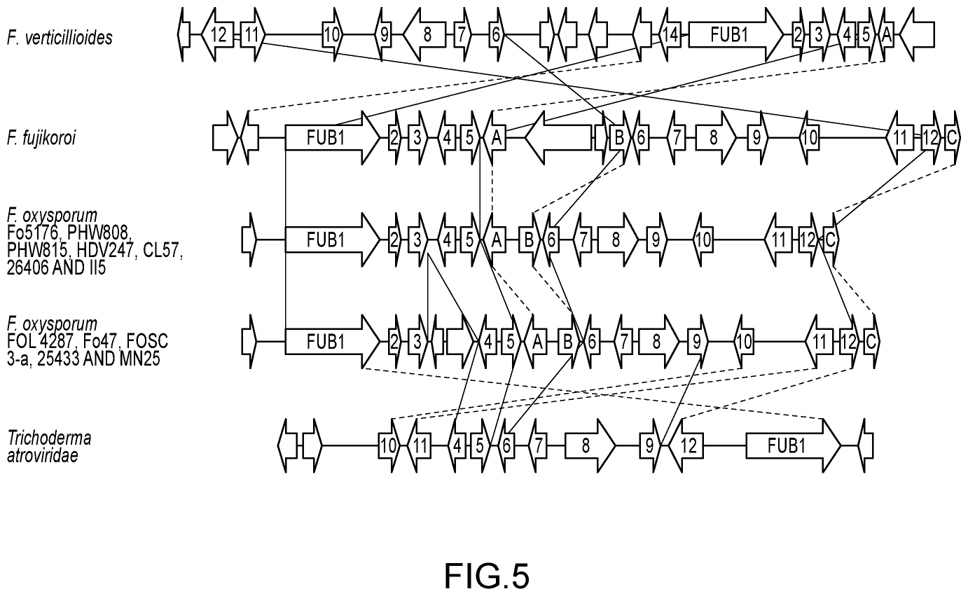

In some embodiments, the recombinant strain comprises a genetic modification of a gene in the fusaric acid biosynthesis pathway and produces reduced levels of fusaric acid. Biosynthesis of fusaric acid is linked to a cluster of 12 genes, which include Fub1, Fub2, Fub3, Fub4, Fub5, Fub6, Fub7, Fub8, Fub9, Fub10, Fub11 and Fub12. As noted above, Fub1 encodes a polyketide synthase. FIG. 5 shows the fusaric acid gene arrangement in several filamentous fungi, namely Fusarium verticillioides, Fusarium fujikuroi, several Fusarium oxysporum strains and Trichoderma atroviridae (Brown et al., 2015). These genes have been isolated from several fungal strains and their sequences determined and known in the art. Although no fusaric acid was detected in MK7, surprisingly analysis of its DNA sequence revealed that MK7 genome possesses a complete set of 12 genes for fusaric acid biosynthesis as in Fusarium fujikuroi. Thus, there is a possibility that MK7 may have a potential to produce fusaric acid under some culture conditions.

The term genetic modification refers to any modification or alteration of the naturally occurring (wild type) nucleotide sequence of the gene that results in reduced expression of the protein encoded by the gene or reduced function of the protein encoded by the gene. The modification may be in the coding sequence, or in the 5′ or 3′ untranslated regions, or the promoter, the terminator, an enhancer, or another regulatory element of the gene. Such modifications may include deletions, insertions, and/or substitutions of one or more nucleotides in the wild type sequence. The genetic modification may cause a disruption of the gene such that the cells of the recombinant strain produce a reduced amount of the protein encoded by the gene. For example, disruption of the Fum1 gene may reduce or prevent or abolish the expression of the functional protein encoded by the Fum1 gene.

The disruption may involve any one or more of the following changes: disrupt the gene sequence preventing transcription of a full length mRNA, interfere with the splicing or editing of the mRNA, interfere with the translation of the mRNA, introduce a stop codon into the encoding sequence to prevent the translation of full-length protein, change the coding sequence of the protein to produce a less active or inactive protein or reduce protein interaction with other proteins, or change the coding sequence of the protein to produce a less stable protein or target the protein for destruction, or cause the protein to misfold or be incorrectly modified (e.g., by glycosylation), interfere with cellular trafficking of the protein, or it may reduce the efficiency of the promoter, or enhancer, or any regulatory element.

In some embodiments, the genetic modification comprises a deletion of the gene. The deletion may be complete or partial. The deletion may be in the coding sequence, the promoter, the terminator, an enhancer, or another regulatory element of the gene. The deletion may be performed by the complete or partial deletion of a portion of the chromosome that includes any portion of the gene.

Methods of making genetic modifications, including techniques of molecular biology (including recombinant techniques), microbiology, cell biology, biochemistry, nucleic acid chemistry, and immunology, are well known to those skilled in the art. Such techniques are explained fully in the literature, such as, Methods of Enzymology, Vol. 194, Guthrie et al., eds., Cold Spring Harbor Laboratory Press; Methods in yeast genetics: a laboratory course manual, Rose et al., Cold Spring Harbor Laboratory Press; Molecular Cloning: A Laboratory Manual, Sambrook et al. and Molecular Cloning: A Laboratory Manual, Sambrook and Russel, (jointly referred to herein as “Sambrook”); Current Protocols in Molecular Biology (F. M. Ausubel et al., including supplements); PCR: The Polymerase Chain Reaction, (Mullis et al., eds.), Current Protocols in Nucleic Acid Chemistry John Wiley & Sons, Inc., New York).

Any of these methods may be used to introduce a genetic modification in the genome of fungal strains. Examples include without limitation, homologous recombination, genome editing techniques such as TALEN (transcription activator-like effector nucleases) or CRISPR (clustered regularly interspaced short palindromic repeats), or site specific recombination using Cre-LoxP or FlpFRT systems.

In some embodiments, the genetic modification of the gene is performed using homologous recombination. In homologous recombination nucleotide sequences are exchanged between two similar or identical molecules of DNA. This phenomenon can be exploited to introduce a genetic modification in a target gene. Thus, foreign DNA with a sequence similar to that of the target gene but flanked by sequences identical to the ones upstream and downstream of the target gene's location is introduced into a cell; the cell recognizes the identical flanking sequences as homologues, causing target gene DNA to be swapped with the foreign DNA sequence during replication. This exchange results in genetic modification, including inactivation or knock out of the target gene.

In some embodiments, the genetic modification of the gene is performed using a genome editing technique such as CRISPR. The CRISPR gene editing system comprises a “guide RNA” (gRNA) which binds to a specific sequence in the target gene and a Cas9 nuclease. When the gRNA and a gene encoding the Cas9 nuclease, or the Cas9 nuclease itself are delivered into a cell, the gRNA binds to the target gene sequence and the Cas9 nuclease cuts the DNA at the target sequence. Once the DNA is cut, the cell's natural DNA repair mechanism can repair the site by adding or deleting nucleotides during which a genetic modification is introduced into the target gene. Other suitable enzymes such as Cpfl may also be used instead of Cas9.

In some embodiments, the genetic modification of the gene is performed using a genome editing technique such as TALEN. This technique uses nucleases generated by fusing a transcription activator-like effector (TALE) DNA-binding domain capable of selective binding to a desired target sequence and a DNA cleavage domain which can cut the DNA strand. The TALE DNA binding domain contains a repeated highly conserved 33-34 amino acid sequence with divergent 12th and 13th amino acids called Repeat Variable Di-residue (RVD). The TALEN technology can be used to edit genomes by inducing double-strand breaks in a target gene sequence which trigger the cell's natural DNA repair mechanisms and can result in insertion or deletion of nucleotides in the target sequence at the break sites, thus introducing genetic modifications in the target genes.

Examples 1 and 2 describe identification and sequencing, and genetic modification of Fum1 gene in an edible filamentous fungal strain. However, it is expressly understood that Fum1 is for exemplary purposes only, and any other gene, such as Fum2, Fum3, Fum6, Fum7, Fum8, Fum10, Fum11, Fum13, Fum14, Fum15, Fum16, Fum17, Fum18, Fum19, Fum20, Fum21, Fus1, Fus2, Fus3, Fus4, Fus5, Fus6, Fus7, Fus8, Fus9, Fub 1, Fub2, Fub3, Fub4, Fub5, Fub6, Fub7, Fub8, Fub9, Fub 10, Fub 11, Fub 12 or any combination thereof, may be similarly genetically modified.

As explained in detail in Example 1, the GenBank database and the literature were searched to identify Fum1 sequences that may be similar to the Fum1 in strain MK7. Fusarium fujikuroi was identified as the most closely related to MK7 with a number of genes of F. fujikuroi having at least about 93% nucleotide homology to genes found in strain MK7. The Fum1 gene in F. fujikuroi (SEQ ID NO:1) was BLASTed against the MK7 genome using the SEED viewer routine on the RAST server (Aziz et al., 2008) to locate the Fuml gene of MK7 including 1000 bp left flanking (5′ end) and right flanking (3′ end) untranslated regions (UTRs). The sequence of the Fum1 gene of MK7 including the 5′ and 3′ UTRs is shown in SEQ ID NO:2.

As further described in Example 2, sequences in the untranslated flanking regions were targeted for homologous recombination (HR). Primers were designed to amplify the MK7 Fum1 gene flanking regions. The flanking regions and a selective marker cassette was prepared for assembly into a pUC19 vector. Since wild type (or non-transformed) strain MK7 is susceptible to hygromycin antibiotics, the selection marker used for MK7 transformants was that associated with hygromycin resistance (hph) which allowed for selection of hygromycin resistant MK7 genetic transformants. However, any other suitable selection marker may be used depending on the fungal strain.

Primers and selective marker cassettes were designed and DNA was assembled into vectors for transformation. The transformation efficiency obtained with the methods described herein, and the targeting sequences and vectors described herein, was surprisingly high. Genotypic results based on presence or absence of amplified DNA using different combinations of primers (internal and external to the insertion region) supported the conclusion that the Fum1 gene was deleted and the hph selective marker was inserted therein. Additionally, DNA sequencing across the insertion region also confirmed deletion in Fum1 and replacement with the selective marker.

As described in Example 3, phenotypic analysis of the recombinant fungus revealed a significant reduction in the level of the mycotoxin fumonisin—to <0.2 ppm which amounted to about 99% reduction. It has been reported that the loss of Fum1 gene can adversely impact the growth of the recombinant strain. (See e.g. Sun et al. 2019) Surprisingly, the growth of the recombinant strain was not affected by genetic modification.

Thus, in some embodiments, the genetic modification comprises a modification of the Fum1 gene. In some embodiments, the Fum1 gene sequence is at least about 60%, 65%, 70%, 75%, 80%, 85%, 90%, 91%, 92%, 93%, 94%, 95%, 96%, 97%, 98% or 99%, or any number in the range of 60%-100% identical to SEQ ID NO:2. In some embodiments, the Fum1 gene is at least about 70% identical to SEQ ID NO:2. In some embodiments, the Fum1 gene is at least about 90% identical to SEQ ID NO:2. In some embodiments, the Fum1 gene is at least about 95% identical to SEQ ID NO:2. In some embodiments, the Fum1 gene comprises SEQ ID NO:2. In some embodiments, the Fum1 gene consists of SEQ ID NO:2. SEQ ID NO:2 refers to the sequence of the Fum1 gene from the Fusarium strain MK7 that has been deposited as ATCC Deposit No. PTA-1069.

While Examples 1 and 2 describe the invention using the edible filamentous fungal strain MK7, it is expressly understood that MK7 strain was used for exemplary purposes only, and any other edible filamentous fungal strain may be used in the present invention.

Suitable filamentous fungal strains that may be used in the present invention include any edible filamentous fungal strains selected from the phyla or divisions of basidiomycota or ascomycota. The phylum (or division) basidiomycota comprises, inter alia, the orders Agaricales, Russulales, Polyporales and Ustilaginales, and the phylum ascomycota comprises, inter alia, the orders Pezizales and Hypocreales. In some embodiments, the filamentous fungus may belong to an order selected from Ustilaginales, Russulales, Polyporales, Agaricales, Pezizales and Hypocreales.

The filamentous fungi of the order Ustilaginales may be selected from the family Ustilaginaceae. In some embodiments, the filamentous fungi of the order Russulales may be selected from the family Hericiaceae. In some embodiments, the filamentous fungi of the order Polyporales may be selected from the families Polyporaceae or Grifolaceae. In some embodiments, the filamentous fungi of the order Agaricales may be selected from the families Lyophyllaceae, Strophariaceae, Lycoperdaceae, Agaricaceae, Pleurotaceae, Physalacriaceae, or Omphalotaceae. In some embodiments, the filamentous fungi of the order Pezizales may be selected from the families Tuberaceae or Morchellaceae. In some embodiments, the filamentous fungus may belong to a family selected from Ustilaginaceae, Hericiaceae, Polyporaceae, Grifolaceae, Lyophyllaceae, Strophariaceae, Lycoperdaceae, Agaricaceae, Pleurotaceae, Physalacriaceae, Omphalotaceae, Tuberaceae, Morchellaceae, Sparassidaceae, Nectriaceae and Cordycipitaceae.

Examples of the species of filamentous fungi include, without limitation, Ustilago esculenta, Hericululm erinaceus, Polyporous squamosus, Grifola fondosa, Hypsizygus marmoreus, Hypsizygus ulmarius (elm oyster) Calocybe gambosa, Pholiota nameko, Calvatia gigantea, Agaricus bisporus, Stropharia rugosoannulata, Hypholoma lateritium, Pleurotus eryngii, Pleurotus ostreatus (pearl), Pleurotus ostreatus var. columbinus (Blue oyster), Tuber borchii, Morchella esculenta, Morchella conica, Morchella importuna, Sparassis crispa (cauliflower), Fusarium venenatum, MK7 (ATCC PTA-10698), Disciotis venosa, Cordyceps militaris, Ganoderma lucidum (reishi), Flammulina velutipes, Lentinula edodes, Ophiocordyceps sinensis. Additional examples include, without limitation, Trametes versicolor, Ceriporia lacerate, Pholiota adiposa, Leucoagaricus holosericeus, Pleurotus djamor, Calvatia fragilis, and Handkea utriformis.

In some embodiments, the filamentous fungus may be a Fusarium species. In some embodiments, the filamentous fungus may be the Fusarium strain MK7 (ATCC PTA-10698 deposited with the American Type Culture Collection, 1081 University Boulevard, Manassas, Va., USA). MK7 was previously reported to belong to a Fusarium oxysporum species. However, subsequent phylogenetic work with various protein sequences has shown that it belongs in the F. fujikuroi species complex. In some embodiments, the filamentous fungus may be Fusarium venenatum.

Genetic modification of one or more genes encoding proteins involved in the fumonisin biosynthetic pathway will result in the suppression or reduction in the level of fumonisins in the recombinant fungal strain. Such strains are economically valuable as they require minimal or no processing to reduce the level of fumonisin toxins.

In some embodiments, the recombinant fungal strain produces, on a dry weight basis, less than about 10 ppm, 9 ppm, 8 ppm, 7 ppm, 6ppm, 5 ppm, 4.5 ppm, 4 ppm, 3.5 ppm, 3 ppm, 2.5 ppm, 2 ppm, 1.5 ppm, or 1 ppm of fumonisins. In some embodiments, the recombinant fungal strain produces less than about 10 ppm of fumonisins on a dry weight basis. In some embodiments, the recombinant fungal strain produces less than about 5 ppm of fumonisins on a dry weight basis. In some embodiments, the recombinant fungal strain produces less than about 2 ppm of fumonisins on a dry weight basis. In some embodiments, the recombinant fungal strain does not produce any functional fumonisins.

The term fumonisin refers to any one or more of all fumonisins including fumonisins A, B, C, and P. Fumonisin B includes FB1, FB2, FB3, and FB4. In some embodiments, the fumonisins comprise fumonisins FB. In some embodiments, the fumonisins comprise fumonisins FB1, FB2, FB3, and FB4. The term fusarins refers to all fusarins, including fusarin A, B, C, D and F. In some embodiments, fusarins comprise fusarin C.

In another aspect, the present invention includes a method to produce the recombinant edible filamentous fungal strains described above. In some embodiments the strains are produced using homologous recombination to introduce genetic modifications. In some embodiments the strains are produced using gene editing systems such as CRISPR or TALEN to introduce genetic modifications.

In another aspect, the present invention includes a filamentous fungal biomass comprising interwoven mycelial and/or hyphal filaments comprising the recombinant edible filamentous fungal strains described herein.

In some embodiments, the filamentous fungal biomass comprising interwoven mycelial filaments is a biomat. As used herein, the term “biomat,” unless otherwise specified, refers to cohesive, homogeneous mats of filamentous fungal tissue comprising a network of interwoven mycelial filaments. Biomats as that term is used herein may be characterized by one or more of a density of between about 25 and about 200 grams dry weight per liter media, a solids content of between about 5 wt % and about 20 wt %, and sufficient tensile strength to be lifted substantially intact from the surface of a growth medium. In some embodiments, the biomats are produced by a surface fermentation method. Such biomats have been described in WO2017/151684, WO 2019/046480 and PCT/US20/20152, the disclosure of each of which is incorporated herein by reference in entirety.

In some embodiments, the filamentous fungal biomass may be produced by the methods described in WO 2019/099474, the disclosure of which is incorporated herein by reference in entirety.

As noted above, it has been reported that disruption of genes involved in fumonisin synthesis affect the growth of the recombinant strain. Surprisingly, as described herein the growth of the recombinant strains was not affected to a discernible degree and the recombinant strains were able to form biomats similar to the unmodified strain.

In another aspect, the present invention includes a method of producing a biomat comprising the recombinant edible filamentous fungal strains described herein. In some embodiments, the method requires inoculating an artificial liquid growth medium with an effective amount of the recombinant edible filamentous fungal strain, incubating the inoculated filamentous fungi to produce a filamentous fungal biomat on the surface of the liquid medium, and harvesting the filamentous fungal biomat.

In another aspect, the present invention includes a method of preparing a food material comprising culturing a biologically pure culture of the recombinant edible filamentous fungal strain of the present invention and harvesting it. In some embodiments, the method comprises inoculating an artificial liquid growth medium with an effective amount of a biologically pure culture of the recombinant edible filamentous fungal strain and incubating the inoculated filamentous fungi to produce a filamentous fungal biomat on the surface of the liquid medium.

In another aspect, the present invention includes food materials comprising the recombinant edible filamentous fungal strains described herein. Edible filamentous fungi grown in a biomat form may contain a surprisingly high protein content, and can be directly used as a protein source. They can be used as meat substitutes, and can be used in the preparation of vegetarian or vegan food products. The food products may comprise the recombinant fungi as the sole source of protein or may be include additional proteins.

The recombinant filamentous fungi can be processed to prepare particles of appropriate sizes. Fine particles in the form of a flour are useful in the preparation of food products such as baked goods, including but not limited to bread, rolls, muffins, cakes, cookies, pies, etc. or can be sprinkled on other food products. Liquid dispersion of recombinant fungal particles may be used as a drink or beverage, including as a substitute for any milk product such as dairy milk, almond milk, rice milk, soy milk etc. It can be used in a number of recipes including soups, ice cream, yogurt, smoothies, fudge, and candies such as caramel and truffles. Large particles mimic the texture and chewiness of meat products such as chicken nuggets or hamburgers and are useful in the preparation of products such as a filler or extender of meat products, or their vegetarian versions.

Filamentous fungal biomats and methods of producing them by surface fermentation, as well as their use in preparing food materials have been described in detail in WO/2017/151684, WO 2019/046480 and PCT/US20/20152, which are incorporated herein their entirety.

In another aspect, the present invention includes durable sheet materials, such as textiles, fabrics and leather analogs, comprising the recombinant filamentous fungal strains described herein. In some embodiments, such materials are obtained by growing the strains into filamentous fungal biomass comprising interwoven mycelial and/or hyphal filaments, e.g. biomats, as described herein and further processing them to impart the desired visual, olfactory and tactile properties, e.g. those associated with animal leather. Such materials and methods of preparing them have been described in U.S. Provisional Patent Application 62/966,525, the disclosure of which is incorporated herein in its entirety. Such methods can generally include preparing a durable sheet material comprising fungal biomass by inactivating a fungal biomass; size reducing the fungal biomass; combining the fungal biomass with at least one component selected from a plasticizer, a polymer, a crosslinker, and a dye to form a blended composition; casting the blended composition to form a cast sheet; removing solvent from the cast sheet; and curing the cast sheet, such as by drying or heat-pressing the cast sheet, to form the durable sheet material. This method may also include adding a natural fiber material or a synthetic material to the blended composition. Alternatively, the method can be conducted without size reducing in which case, there is no need to cast the fungal biomass material.

The recombinant filamentous fungal strains described herein can also be used to prepare multilayer or composite materials. In some embodiments, the fungal strain may be grown in a nutrient material containing particulate material, such that the mycelial filaments grow around the particulate material bonding it together to form a structural material. In some embodiments, the fungal strain may be grown in a nutrient material through a porous material. In some embodiments, the fungal strain may be grown in a mold or an enclosure and mechanically compressed to form a self-supporting structural material. In some embodiments, the fungal strain may be grown on or around a scaffold or lattice to form a structural material.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims. The examples and figures are provided for the purpose of illustration only and are not intended to limit the scope of the present invention. Each publication or other reference disclosed herein is incorporated herein by reference in its entirety, to the extent that there is no inconsistency with the present disclosure.

### EXAMPLES

### Example 1. This Example Explains the Isolation of the Fum1 Gene Sequence from Filamentous Fungal Strain MK7

Bioinformatic methods were used to identify and assess the Fum1 gene region of strain MK7, including the exons and introns within the coding region, as well as the flanking 5′ and 3′ untranslated regions (UTRs).

The GenBank database (Benson et al., 2012, Altschul et al., 1997) as well as the literature was searched to identify Fum1 sequences in organisms most closely related to strain MK7 (e.g., Fusarium fujikuroi and Fusarium oxysporum). Annotated sequences from F. fujikuroi were found to be most desirable since F. fujikuroi strains are the most closely related to strain MK7 and many genes in F. fujikuroi have greater than 93% nucleotide homology to genes in strain MK7. The Fum1 gene sequence including exons and introns from F. fujikuroi IMI 58289 starting at the ATG start codon at the 5′ end and ending with the TGA stop codon is shown as SEQ ID NO:1. This sequence was BLASTed against the MK7 genome using the “SEED viewer” routine in the RAST server (Aziz et al., 2008) to identify Fum1 gene in MK7.

The MK7 Fum1 gene sequence including 1000 bp left flanking (5′ end) and right flanking (3′ end) untranslated regions (UTRs) is shown as SEQ ID NO:2.

### Example 2. This Example Explains the Construction of a Recombinant Modified MK7 Strain Comprising a Disruption in the Fum1 Gene

### Construction of a Vector for Fum1 Gene Disruption by Homologous Recombination

The Fum1 disruption vector was prepared using standard molecular biology procedures. The vector included a DNA sequence having a 815 nt region homologous to the DNA sequence spanning part of the 5′ untranslated region (Left Flank) and a DNA sequence having a 821 nt region homologous to the DNA sequence spanning part of the 3′ untranslated region (Right Flank). These sequences were designed to target the Fum1 gene and replace the region of the genome between the Left and Right Flanks with an intervening cassette sequence. The intervening cassette included a hph (hygromycin resistance) selection marker.

Primers to PCR amplify the desired homologous recombination regions (815 and 821 nt) and the selective marker cassette were designed using the standard primer design strategies outlined in the Premier Biosoft website: http://www.premierbiosoft.com/tech_notes/PCR_Primer_Design.html. Primers were checked for hairpins, self-complementary and self-annealing using the Oligo Calc, Oligonucleotide Properties Calculator web site: http://biotools.nubic.northwestern.edu/OligoCalc.html. The primer sequences used herein are shown in Table 1.

### Homologous Recombination Target Regions

Sequences in the untranslated flanking regions were used as targets for homologous recombination (HR). The flanking regions used for HR of the Fum 1 gene in strain MK7 (5′ to 3′ direction) are shown below, where underlined/italicized sequence are primers (with labels in parenthesis).

MK7 Homologous Recombination-Left Flank
(5′ end) Untranslated Region (UTR)
(SEQ ID NO: 3) [symbol]
GCTCTTGCGCTACGCTATAGTGTAATTTAC
CCACCGGTAGCCAACTCTTCAAGCCTCTAATCATTCGAGTCCTCCTAACC
ACCTCAGTGTTCCGCTAAGCGCAAATTGGCAGGTGGCAATACCGGCATGC
GCAATCATGTCATGTAGTGCATATACCCGCCGTGGGGATCTTAGCCATTT
TTCTTCCGCCATATGTGCCGTGTTTTCCGAGTCCGACACCATGATCCACG
GCAGACTTGCTCCTAAGAACTTTGCTCGTGACACGGCAAACGATAAAGGA
CAACCGTCTCCGTGCAGGTCAAAATACGGCTTCGGCATCTACAAATAGAA
ATCCGATGTGGTCCAAATACGAAGAAGCCGTCATGATAGATACTGGTCTG
AATCGGACATCCGAAGCCTGGCGATTACGTTAATAACCACGGCCCCATAC
CTGCGCGTTAGATCAAACCGGATTCTGGTTCTGTAATTAGTCAATGGGGC
AAATTCCCCTTTGCCCGATCCGATTGGTTCCGTGGGTGTTGATTGTACTC
AGACATCACTGTACTTGACCTGAGGATTCGTGTACTAGCATTTTGGCAAG
CAGAAGAGAGCAATGGACAGTCTACTCGGTTCTTCATCACGGAACATGTC
ATTTGTCTAAGGGGACTGTGATTATTGTGCATCCGAATGCGGCGTGTTCT
CTCGGATGGGAACTCCATAGCGTCTAGATTACCGAAGTATCGATGGTGGT
TGTAGTTCAGTTATGATACATAAGCATCATAAGATCAAAAATATCATCCG
ACCAAGCCTTCGTAGCT [symbol]
MK7 Homologous Recombination-Right Flank
(3′ end) Untranslated Region (UTR)
(SEQ ID NO: 4) [symbol]
AGGTACTACTTAACTTGAGCCTTT
ACATACTTAAATACATCTCGAAAATGCTCCTGAATTATACGCTCGTATGT
GCATGTGCCCTATAATGATGATTCCTTACTACTGAGCCTCTACTTTCGCC
AACAGACTGGCCCACAGTAGTTGTCCCCTGTATCACTGTATTCTGGCGGA
CACCCATATCGAACTTGTTCAACTCGTTAAAGGCGTCCTATATTTTTCAG
AGCACGGCAATAATAAGTTTCTGAGTCTGTGCCATACAACCCGCTATTAC
AGCGGAGCCTAATAATTCATTCATCACAATGCTGTTTCCACCACTGCGCA
ACCAGTGTCTTCATTTCTGGCGAAGGTAAATAGAATGAATACATAACATA
GCTACCCCCAGAATGTCCATTCTAACTACACATAAACTTCTTCTACATAT
CTCCCCTTGTCCAACCCTGCTCTCCATTCTACAACCGTCTTTACTGCTGT
TTCCCCTCTGTTCTGATCAGTTTCCAATAGCATCGGCCCTAGAACCTCTT
GCACGTCATTTGCCATTTTCTTACCTCCGCAGACAAGGACCTTGGCGCCA
GCCACCCAGAGGCGAACAATTTCATCTCGACATCTTTCGAGCTTCTCAGT
AACGTACCTGCAGCCTTGGGCACCTGTGGCAGAAGGGTCACGGCTATAGG
CTCTTCGCATTTGCACGATGCCCATGGCTTCGTAGGCTTCCATTTCAATC
GAGTACAGAGAGTCAAGCCCGGGTCCTCGACAACCGAAAAACAGGAGGGC
CGGCCCAAGCTTCGATTTGGA

[symbol]

### Hygromycin Selective Marker and HPH Cassette

The selective marker used for selection of MK7 transformants was hygromycin resistance (hph). Wild type strain MK7 (non-transformed strain) is susceptible to the hygromycin antibiotic in that hygromycin inhibits growth and kills MK7. However, genetic transformation of wild type MK7 by insertion of the hph gene with its flanking promoter/terminator sequences as a cassette was expected to impart antibiotic resistance. The hph gene preceded by a anTRPCpro promotor and followed by a anTRPCter terminator was derived from Aspergillus nidulans. The pFC332 vector (plasmid) in A. nidulans that encodes the hygromycin cassette was provided by Christina Spuur Nodvig and Uffe Hasbro Mortensen at the Technical University of Denmark. The sequence of the full selective marker cassette (anTRPCpro-hph-anTRPCter) is shown below, where underlined/italicized sequences are primer sites with labels in parenthesis, the ATG start codon and the TAG stop codon for the hph gene are shown in bold, and the greyed sequences are the anTRPCter and the anTRPCpro sequences, respectively:

hph Cassette
(SEQ ID NO: 5) [symbol]
aatgctattttggtttagtcgtccagg
cggatcacaaaatttgtgtcgtttgacaagatggttcatttaggcaactg
gtcagatcagcccacttgtaagcagtagcggcggcgctcgaagtgtgact
cttattagcagacaggaacgaggacattattatcatctgctgcttggtgc
acgataacttgtgcgtttgtcaagcaaggtaagtgaacgacccggtcata
ccttcttaagttcgcccttcctccctttatttcagattcaatctgactta
cctattctacccaagcatccaaATGcctgaactcaccgcgacgtctgtcg
agaagtttctgatcgaaaagttcgacagcgtctccgacctgatgcagctc
tcggagggcgaagaatctcgtgctttcagcttcgatgtaggagggcgtgg
atatgtcctgcgggtaaatagctgcgccgatggtttctacaaagatcgtt
atgtttatcggcactttgcatcggccgcgctcccgattccggaagtgctt
gacattggggagttcagcgagagcctgacctattgcatctcccgccgtgc
acagggtgtcacgttgcaagacctgcctgaaaccgaactgcccgctgttc
tggagccggtcgcggaggccatggatgcgattgctgcggccgatcttagc
cagacgagcgggttcggcccattcggaccgcaaggaatcggtcaatacac
tacatggcgtgatttcatatgcgcgattgctgatccccatgtgtatcact
ggcaaactgtgatggacgacaccgtcagtgcgtccgtcgcgcaggctctc
gatgagctgatgctttgggccgaggactgccccgaagtccggcacctcgt
gcacgcggatttcggctccaacaatgtcctgacggacaatggccgcataa
cagcggtcattgactggagcgaggcgatgttcggggattcccaatacgag
gtcgccaacatcttcttctggaggccgtggttggcttgtatggagcagca
gacgcgctacttcgagcggaggcatccggagcttgcaggatcgccgcgcc
tccgggcgtatatgctccgcattggtcttgaccaactctatcagagcttg
gttgacggcaatttcgatgatgcagcttgggcgcagggtcgatgcgacgc
aatcgtccgatccggagccgggactgtcgggcgtacacaaatcgcccgca
gaagcgcggccgtctggaccgatggctgtgtagaagtatcgccgatagtg
gaaaccgacgccccagcactcgtccgagggcaaaggaaTAGagtagatgc
cgaccgatccccactaccgcattaagacctcagcgcggccgcaaatttaa
ataaaatgaagtgaagttcctatactttctagagaataggaacttctata
gtgagtcgaataagggcgacacaaaatttattctaaatgcataataaata
ctgataacatcttatagtttgtattatattttgtattatcgttgacatgt
ataattttgatatcaaaaactgattttccctttattattttcgagattta
ttttcttaattctctttaacaaactagaaatattgtatatacaaaaaatc
ataaataatagatgaatagtttaattataggtgttcatcaatcgaaaaag
caacgtatcttatttaaagtgcgttgcttttttctcatttataaggttaa
ataattctcatatatcaagcaaagtgacaggcgcccttaaatattctgac
aaatgctctttccctaaactccccccataaaaaaacccgccgaagcgggt
ttttacgttatttgcggattaa

[symbol]

[symbol]

TABLE 1
Primers used for amplifying left and right homologous recombination
regions (HR-L and HR-R) and the hph cassette, for assembling the
shuttle vector containing pUC19 backbone harboring amp resistance
with the HR-L and HR-R regions and with the hph cassette, and for
checking/confirming HR results in mutant strains. Primers used for
assembling the vector contained tail regions for bridging components
of the vector. The non-tail portions of the tailed primers are
delineated by underlining.

| SEQ ID | | |
|---|---|---|
| NO. | Oligo Name | Sequence (5′ to 3′) |
| SEQ ID NO: | HR-L_Forward | GAGCTCGGTACCCGGGGATCC [symbol] |
| 6 | | |
| SEQ ID NO: | HR-L_Forward_S | [symbol] |
| 7 | | |
| SEQ ID NO: | HR-L_Reverse | CGAGTATAAGGTTACGGTGGATG |
| 8 | | |
| SEQ ID NO: | HPH_Forward | CCACCGTAACCTTATACTCG [symbol] |
| 9 | | |
| SEQ ID NO: | HPH_Forward_S | [symbol] |
| 10 | | |
| SEQ ID NO: | HPH_Reverse | CTGTTATCTTGTTCAGTGTT [symbol] |
| 11 | | |
| SEQ ID NO: | HPH_Reverse_S | [symbol] |
| 12 | | |
| SEQ ID NO: | HR-R_Forward | AACACTGAACAAGATAACAGATTCTG |
| 13 | | |
| SEQ ID NO: | HR-R_Reverse | CAGGTCGACTCTAGAGGATC [symbol] |
| 14 | | |
| SEQ ID NO: | HR-R_Reverse_S | [symbol] |
| 15 | | |
| SEQ ID NO: | FUM5_5′UTR_F0b | CATCATGGCTCACAGAGAATTTCAT |
| 16 | | |
| SEQ ID NO: | FUM5_5′UTR_F0 | GCGTTTGCTGGTGATCTTG |
| 17 | | |
| SEQ ID NO: | MK7_fum5_exon_1_F1 | ATGGTAGTAATCGAATCACCC |
| 18 | | |
| SEQ ID NO: | MK7_fum5_exon_1_R1 | CGGGGGTGATTCGATTACTA |
| 19 | | |
| SEQ ID NO: | FUM5 exon 3 F1 | AACGATTCAAGGCATACCAAGG |
| 20 | | |
| SEQ ID NO: | FUM5 exon 3 R1 | CCTTGGTATGCCTTGAATCGTT |
| 21 | | |
| SEQ ID NO: | MK7_fum5_exon_7_F1 | TAACACTAGACGGGCTCAAGA |
| 22 | | |
| SEQ ID NO: | FUM6_3′_R0 | CTTGTTCAGCTTCGGCCAA |
| 23 | | |
| SEQ ID NO: | FUM6_3′_R0b | AGCGCATGCTGAAAGACAGT |
| 24 | | |

### Transformation of MK7

The following procedure was used to assemble DNA into a vector for transformation into strain MK7. The 5′ HR-L and 3′ HR-R flanking regions, and selective marker hph cassette were each amplified in an individual PCR reaction using high-fidelity (proof-reading) taq polymerases PrimeStar Max (Takara Bio Inc. Kusatsu, Japan) or pfx Phusion™ (Thermo-Fisher, Waltham, Mass.) using the appropriate primers and following the instructions and conditions specific to the polymerase. After PCR amplification, the products were run on a 1% agarose-TAE gel (containing 0.04 μl/ml ethidium bromide) in TAE buffer. Bands corresponding to the correct size on the 1% agarose-TAE gel were visualized on a UV gel box and excised using a new, clean, flame-sterilized razor blade. Following excision, bands were dissolved and purified as described in the DNA gel purification kit (Zymoclean Gel DNA Recovery Kit, Cat# D4007, Zymo Research, Irvine, Calif.). Purified DNA was quantified on a NanoDrop™ (Thermo-Fisher).

Purified and quantified DNA was combined with linearized vector (Takara pUC19 control vector) following the commercial instructions for restriction enzymes. In-Fusion (Takara Bio Inc.) reaction conditions to fuse the DNA fragments were followed as per instructions for the In-Fusion kit (In-Fusion HD Cloning Kit User Manual). A 96-well format PCR thermal cycler was used for the incubation and cooling steps.

After cooling, the In-Fusion products were transformed directly into Stellar™ Competent E. coli (Takara Bio Inc.) as described by Protocol PT5055-2 for Stellar Competent Cells. Alternatively, In-Fusion reactions can be stored at −20° C. for future use. Excess reaction mix was stored at −20° C. for additional transformations when needed. Transformed E. coli were plated onto selective media LB-plates containing 100 μg/ml ampicillin. Plates were allowed to incubate overnight in a 37° C. incubator. Colonies were screened for presence of the appropriate DNA fragments using the short versions of the primers (not containing tails) by colony PCR as follows.

Colonies were picked using a sterile toothpick or pipette tip, tapped to a marked grid on a fresh “master plate” (LB plate containing the antibiotic) and then swirled in 50-100 μl sterile deionized H₂O. A portion of this sterile water (dependent on the PCR volume, typically 40-50% of the total PCR volume) containing resuspended cells was then used as template in a PCR reaction. While the PCR's were run on the thermal cycler, the “master plate” were incubated at 37° C. Typically, colonies began to form on this plate in a matter of hours and were picked the same or following day.

Colonies on the “master plate” that passed the colony PCR screen were grown in 5 ml LB media cultures containing 100 μg/ml Ampicillin. Cultures were grown overnight at 37° C. and 250 rpm shaking. The following day, frozen stocks were prepared by mixing 800 μl of overnight culture with 200 μl of filter sterilized 80% glycerol in water. The remaining culture was harvested by centrifugation and a plasmid mini-prep was performed following the manufacturer's instructions (ZymoPURE Plasmid Miniprep Kit, Cat# D4211, Zymo Research). Purified plasmid DNA in deionized H2O was quantified using a NanoDrop™ (Thermo-Fisher). Quantified plasmids were then prepared for Sanger sequencing using an array of primers that sufficiently covered the inserts in the host vector (pUC19). Typically, a single Sanger sequencing read is good for 800-1000 nucleotides. Sequencing was done at the Iowa State University DNA facility. The resultant .ab1 files were analyzed using BioEdit sequence analysis software (version 7.2.6.1-2017; Hall, 1999). Sequences were BLASTed within the RAST server or aligned to other sequences using BioEdit alignment software.

The Plasmid MAXI-prep kit was used to generate enough DNA for transformations into MK7 (ZymoPURE Plasmid Maxiprep Kit #D4202, Zymo Research). Colonies on the master plate corresponding to plasmids that passed the sequencing analysis from above were propagated in 50-100 ml of LB cell culture medium. Cells were harvested by centrifugation and plasmids were isolated using a plasmid MAXI-prep kit. Purified DNA was suspended in deionized H2O as recommended in the kit instructions and quantified using NanoDrop (Thermo-Fisher).

To isolate single colony forming units (CFUs), wild-type strain MK7 from frozen stocks prepared previously were serially diluted in sterile 0.02 M NaCl water were streaked onto YPD agar (10 g yeast extract, 20 g peptone, 20 g dextrose per 1 L and 15 g agar; dextrose added as a filtered 20% solution; other reagents were sterilized by autoclaving) plates containing 100 μg/ml ampicillin. Plates were incubated for 3-5 days until colonies were observed. The resultant cells were scraped from a single colony with a sterile wooden tooth pick and used to inoculate 50 ml of liquid YPD culture (containing 100 μg/ml ampicillin). The culture was grown in a 250 ml shaker flask for 3-4 days at 30° C. with 250 rpm orbital shaking. After incubation, the culture was filtered through a 45 micron polypropylene filter (Cat #PP4504700, Millipore, Burlington, Mass.), which allowed microconidia to pass through while filtering out hyphae. The filtrate was used to inoculate fresh liquid YPD media (containing 100 μg/ml ampicillin) at a ratio of 1:10. This culture was then grown overnight at 30° C. with 250 rpm orbital shaking. The following morning, cells were harvested by filtration onto sterile 0.2 micron filter membrane (Millipore).

For protoplast preparation, enzyme solution was made by adding 25 mg/ml driselase (Cat #D8037, Sigma-Aldrich, St. Louis, Mo.) and 20 mg/ml lysing enzymes (Cat #L3768, Sigma-Aldrich) to sterile deionized H₂O. This solution was incubated at room temperature for 30 minutes while shaking at 200 rpm. After incubating, an equal volume of 2 M KCl solution was added and then filtered through a 0.22 micron syringe filter to remove insoluble driselase and lysing enzymes. This solution represented the stabilized protoplasting solution (12.5 mg/ml driselase and 10 mg/ml lysing enzymes in 1 M KCl). The stabilized protoplasting solution was used to resuspend the collected cells to a concentration of ˜25 mg cells per ml. The culture was incubated for 3-4 hour at 30° C. with 200 rpm orbital shaking. After incubation, the protoplasts were separated from mycelial debris by filtration through 45 micron nylon filter, washed twice with STC buffer (1.3 M sorbitol, 10 mM Tris, pH 7.5, 50 mM CaCl₂) and resuspended in STC buffer at a concentration of 2×10⁵ cells per ml (as defined in the next step).

Cell counts were determined using a hemocytometer as follows. 10 ul of cell suspension was pipetted to the edge of the slide cover. The cell suspension was diluted when necessary. Using the hemocytometer grid, cells were counted under 300× magnification until at least 100 cells were counted. Counting was done using a clicker counter. The number of cells per ml of suspension was calculated as per hemocytometer instructions:

$$Total cells / ml = Total cells counted \times \frac{dilution factor}{\# of Squares} \times 10 , 000 cells / ml$$

To prepare the pUC19 vector for transformation, 20 μg of the vector (prepared earlier) was dried down completely using a speed-vac. The dried down vector was resuspended in 50 μl of STC and 50 μl of 25% PEG 6000, 50 mM of CaCl2, 10 mM of Tris-HCl, pH 7.5.

Transformations were conducted by mixing 100 μl of protoplast suspension with 100 μl of prepared vector suspension from above and incubated at room temperature for 20 minutes. A water control transformation was included where only deionized sterile water and no vector was added to the mix. After this 20 minute period, the protoplast suspensions were plunged into 1 ml of PEG solution, mixed for several seconds before diluting with 2 ml of STC.

After transformation, all 3.2 ml of the cell and DNA mixture were plated onto RM plates (30 g yeast extract, 30 g tryptone, 200 sucrose, 15 g agar per 1 L media) containing 200 μg per ml hygromycin. Volumes of 400 μl of the mixture were spread onto each plate and the liquid was allowed to dry and/or soak into the plate. Control protoplasts were treated as above but without the addition of plasmid. Control reactions were also plated onto non-selective (no hygromycin) RM-agar plates to test for cell viability. Transformants were observed after a 5-day incubation at 30° C.

### Genotyping

To genotype the transformants, cells from single colonies were picked from the RM plates and grown in 5 ml YPD liquid media containing 100 μg/ml ampicillin and 200 μg/ml hygromycin. Cells were collected by centrifugation at 3400×g for 10 minutes. The pellet/slurry was used for DNA extraction. Genomic DNA was extracted using the FastDNA Spin Kit for Soil extraction kit (MP Biomedicals, Solon, Ohio) and 500 μl of the cell pellet/slurry. Genomic DNA was quantified by NanoDrop (Thermo-Fisher, Waltham, Mass.). Genomic DNA from wild type and transformed strains were used as templates for PCR screening using primers both internal and external to the inserted selective marker cassette. Primers flanking the recombination event used the high-fidelity polymerase pfx Phusion™ (Thermo-Fisher) to PCR amplify the entire region including flanking unmodified DNA as well as the selective marker cassette. The bands resulting from PCR amplification were visualized on an agarose gel and excised and purified as described above. The purified DNA fragments were analyzed by Sanger sequencing (as described above) using appropriate primers. Sanger sequencing data was then assessed as described above for the proper insertion of the selective marker cassette into the MK7 genome.

All genotypic results based on presence or absence of amplified DNA product as well as product size using different combinations of primers internal and external to the insertion region supported the conclusion that the Fum1 gene was deleted and replaced with the hph selective marker cassette in mutant strains A8b-Fum1 and A9c-Fum1. Additionally, DNA sequencing across the insertion region confirmed deletion of Fum1 and replacement with the selective marker.

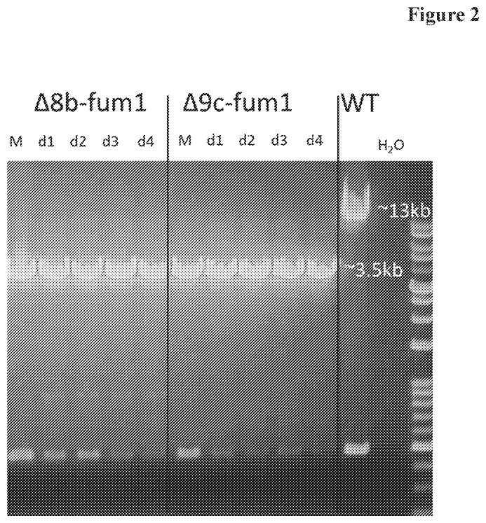

FIG. 2 shows agarose gel band positions showing that the ˜13 kb band expected for the Fum1 harboring wild type (WT) strain was not present in the mother (M) and daughter (d) colonies of the Δ8b-Fum1 and Δ9c-Fum1 mutant strains. The PCR products were generated using HR-L_Forward_S and HR-R_Reverse_S primers, which flank the original Fum1 gene or the expected hph cassette insert. Expected band size for the amplified DNA using these primers is ˜13 kb for the WT strain with Fum1 and ˜3.5 kb for the mutant strains with the hph cassette. The bands from the daughter colony strains (d1) were excised and sequenced to confirm Fum1 deletion and replacement with the hygromycin resistance cassette.

### Example 3. This Example Explains the Phenotypic Analysis of the Recombinant MK7 Fungus Comprising a Disruption in the Fum1 Gene

Fumonisin production by the recombinant (mutant) and wild type strains was evaluated in biomats grown in MK7-102 medium in 204 cm² Pyrex glass trays. After five days of growth, biomats were harvested and dried at 99° C. overnight (20 h), and the fumonisin concentrations in the dried biomass were measured using the VICAM Vertu V lateral flow test system with the Fumo-V Aqua fumonisin analysis kit following the manufacturer's instructions (Vicam, Milford, Mass.), with exception that the samples were vortexed for 10 minutes instead of 1 minute.

Fumonisin concentrations for mutant strains in biomats grown in trays were all below the detection limit for the VICAM system (<0.2 ppm) while the level in the wild type strain was 19 ppm. Visual inspection of the biomats did not reveal any obvious differences among mutant and wild type strains.

TABLE 2
Values of pH, weight and fumonisin measured for mutant and wild type
strain biomats grown in 204 cm² trays.

| | | | | | | Mean | Std dev | | Dry |
|---|---|---|---|---|---|---|---|---|---|
| | | pH | pH | Wet | Dry | dry | dry | | weight |
| | pH | (mat, | (mat, | weight | Weight | weight | weight | Fumonisins | per 0.25 |
| Sample ID | (liquid) | top) | bottom) | (g) | (g) | (g) | (g) | (ppm) | m² |
| Δ8b-fum1-d1 | 4.2 | 7.5 | 4.8 | 6.99 | 2.28 | | | <0.2 | 27.6 |
| Δ8b-fum1-d2 | 5.7 | 7.1 | 5.3 | 12.49 | 3.52 | | | <0.2 | 42.7 |
| Δ8b-fum1-d3 | Contaminated | | | | | | | | |
| Δ8b-fum1-d4 | 5.4 | 7.7 | 5.7 | 11.47 | 3.48 | 3.09 | 0.71 | <0.2 | 42.3 |
| Δ9c-fum1-d1 | 4.6 | 7.6 | 4.5 | 6.08 | 2.00 | | | <0.2 | 24.3 |
| Δ9c-fum1-d2 | 4.4 | 7.4 | 4.4 | 8.29 | 2.65 | | | <0.2 | 32.1 |
| Δ9c-fum1-d3 | 4.3 | 7.9 | 5.1 | 9.41 | 2.70 | | | <0.2 | 32.8 |
| Δ9c-fum1-d4 | 4.3 | 7.8 | 4.2 | 8.29 | 2.46 | 2.45 | 0.32 | <0.2 | 29.8 |
| WT | 4.4 | 7.7 | 5.1 | 11.03 | 3.67 | 3.67 | | 19.0 | 44.5 |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims. The examples are provided for the purpose of illustration and are not intended to limit the scope of the present invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Unless defined otherwise, all technical and scientific terms used herein have the same meaning as commonly understood by one of ordinary skill in the art to which this invention belongs. Any methods and materials similar or equivalent to those described herein can also be used in the practice or testing of the present invention. Each publication, sequence or other reference disclosed below and elsewhere herein is incorporated herein by reference in its entirety, to the extent that there is no inconsistency with the present disclosure.

### REFERENCES

1. Altschul et al., Gapped BLAST and PSI-BLAST: a new generation of protein database search programs, Nucleic Acids Res. 25:3389-3402 (1997).
2. Aziz et al., The RAST Server: rapid annotations using subsystems technology, BMC Genomics 9:75 (2008).
3. Benson et al., GenBank. Nucleic Acids Research 40 (Database-Issue): 48-53 (2012)
4. Hall, T. A, BioEdit: a user-friendly biological sequence alignment editor and analysis program for Windows 95/98/NT. Nucl. Acids. Symp. Ser. 41:95-98 (1999).
5. Huffman et al., Recent advancements in the biosynthetic mechanisms for polyketide-derived mycotoxins. Biopolymers 93(9):764-76 (2010).
6. Visentin et al., Transcription of genes in the biosynthetic pathway for fumonisin mycotoxins is epigenetically and differentially regulated in the fungal maize pathogen Fusarium verticillioides. Eukaryotic Cell 11(3): 252-59 (2012).
7. Medina et al., Integrating toxin gene expression, growth and fumonisin B₁ And B₂ production by a strain of Fusarium verticillioides under different environmental factors. Journal of Royal Society Interface 10:20130320 (2013).
8. Rocha et al., FUM gene expression profile and fumonisin production by Fusarium verticillioides inoculated in Bt and non-Bt maize. Front. Microbiol. 6:1503 (2016).
9. Flaherty and Woloshuk, Regulation of fumonisin biosynthesis in Fusarium verticillioides by a Zinc binuclear cluster-type gene, ZFR1. Appl. Environ. Microbiol. 70(5): 2563-69 (2004).
10. ApSimon, Structure, synthesis, and biosynthesis of Fumonisin B1 and related compounds. Environmental Health Perspectives 109(2): 245-49 (2001).
11. Munkvold and Desjardins, Fumonisins in maize: can we reduce their occurrence? Plant Disease 81(6):556 (1997).
12. Sun et al., Effects of disruption of five FUM genes on fumonisin biosynthesis and pathogenicity in Fusarium proliferatum. Toxins 11:327 (2019).
13. Wiemann P. et al., Deciphering the cryptic genome: genome-wide analyses of the rice pathogen Fusarium fujikuroi reveal complex regulation of secondary metabolism and novel metabolites. PLoS Pathog. 9(6), e1003475 (2013).
14. Niehaus et al., Genetic manipulation of the Fusarium fujikuroi fusarin gene cluster yields insight into the complex regulation and fusarin biosynthetic pathway. Chemistry & Biology 20, 1055-1066 (2013).
15. Brown et al., Identification of a 12-gene fusaric acid biosynthetic gene cluster in fusarium species through comparative and functional genomics. Mol. Plant Microbe Interact. 28, 319-332 (2015).
16. Diaz-Sánchez et al., Identification and regulation of fusA, the polyketide synthase gene responsible for fusarin production in Fusarium fujikuroi. Appl Environ Microbiol 78:7258 (2012)
17. Proctor et al., Co-expression of 15 contiguous genes delineates a fumonisin biosynthetic gene cluster in Gibberella moniliformis. Fungal Genetics and Biology 38, 237-249 (2003).
18. Song et al., Fusarin C biosynthesis in Fusarium moniliforme and Fusarium venenatum. ChemBioChem 5, 1196-1203 (2004).
19. Proctor et al., A polyketide synthase gene required for biosynthesis of fumonisin mycotoxins in Gibberella fujikuroi mating population A. Fungal Genet. Biol. 27:100-112 (1999).
20. Desjardins et al., FUM1 -A gene required for fumonisin biosynthesis but not for maize ear rot and ear infection by Gibberella moniliformis in field tests. Mol Plant Microbe Interact. 15(11):1157-64 (2002).
21. Bacon et al., Production of fusaric acid by Fusarium species. Appl. Environ. Microbiol. 62(11): 4039-43 (1996).
22. Nemudry et al., TALEN and CRISPR/Cas genome editing systems: tools of discovery. Acta Naturae. 2014 July-September; 6(3): 19-40 (2014).

1. A recombinant filamentous fungal strain, wherein the recombinant strain comprises a genetic modification of a gene in a mycotoxin biosynthesis pathway and wherein the recombinant strain produces a reduced level of the mycotoxin.

2. The recombinant filamentous fungal strain of claim 1, wherein the mycotoxin is a polyketide.

3. The recombinant filamentous fungal strain of claim 1, wherein the mycotoxin is selected from the group consisting of a fumonisin, a fusarin, fusaric acid and a combination thereof.

4. The recombinant filamentous fungal strain of claim 1, wherein the recombinant strain comprises a modification of a gene encoding a polyketide synthase.

5. The recombinant filamentous fungal strain of claim 4, wherein the gene encoding a polyketide synthase is selected from the group consisting of Fum1, Fus1, Fub1 and a combination thereof.

6. The recombinant filamentous fungal strain of claim 5, wherein the recombinant strain comprises a modification of the Fum1 gene and the recombinant strain produces a reduced level of fumonisin; or the recombinant strain comprises a modification of the Fus1 gene and the recombinant strain produces a reduced level of Fusarin C; or the recombinant strain comprises a modification of the Fub1 gene and the recombinant strain produces a reduced level of Fusaric acid; or a combination thereof.

7. The recombinant filamentous fungal strain of claim 1,
wherein the mycotoxin is fumonisin and the genetic modification comprises a modification of one or more of the genes selected from the group consisting of Fum1, Fum2, Fum3, Fum6, Fum7, Fum8, Fum10, Fum11, Fum13, Fum14, Fum15, Fum16, Fum17, Fum18, Fum19, Fum20 and Fum21; or
wherein the mycotoxin is fusarin and the genetic modification comprises a modification of one or more of the genes selected from the group consisting of Fus1, Fus2, Fus3, Fus4, Fus5, Fus6, Fus7, Fus8 and Fus9; or
wherein the mycotoxin is fusaric acid and the genetic modification comprises a modification of one or more of the genes selected from the group consisting of Fub1, Fub2, Fub3, Fub4, Fub5, Fub6, Fub7, Fub8, Fub9, Fub10, Fub 11 and Fub12;
or a combination thereof.

8. (canceled)

9. The recombinant filamentous fungal strain of claim 1, wherein the genetic modification of the gene comprises a partial or complete deletion of the gene.

10. The recombinant filamentous fungal strain of claim 1, wherein the genetic modification of the gene is performed using at least one of homologous recombination, CRISPR, and TALEN.

11-12. (canceled)

13. The recombinant filamentous fungal strain of claim 1, wherein the genetic modification of the gene is performed in combination with introducing a selectable marker.

14. The recombinant filamentous fungal strain of claim 13, wherein the selectable marker is hygromycin resistance.

15. (canceled)

16. The recombinant filamentous fungal strain of claim 1, wherein the genetic modification comprises a modification of the Fum1, Fum6 or Fum8 gene.

17. The recombinant filamentous fungal strain of claim 16, wherein the genetic modification comprises a modification of the Fum1 gene.

18. The recombinant filamentous fungal strain of claim 17, wherein the Fum1 gene is at least 70% identical to SEQ ID NO: 2.

19. (canceled)

20. The recombinant filamentous fungal strain of claim 17, wherein the Fum1 comprises SEQ ID NO: 2.

21. The recombinant filamentous fungal strain of claim 1, wherein the filamentous fungus is a Fusarium species.

22. The recombinant filamentous fungal strain of claim 21, wherein the filamentous fungus is selected from the group consisting of MK7 (ATCC PTA-10698) and Fusarium venenatum.

23. (Canceled)

24. The recombinant filamentous fungal strain of claim 1, wherein the recombinant strain comprises less than about 10 ppm of a mycotoxin selected from the group consisting of fumonisin, fusarin or fusaric acid and a combination thereof on a dry weight basis.

25-26. (canceled)

27. The recombinant edible-filamentous fungal strain of claim 1, wherein the fumonisin comprises funonisin FB.

28. (canceled)

29. The recombinant filamentous fungal strain of claim 1, wherein the fusarin comprises fusarin C.

30. A biomat comprising the recombinant filamentous fungal strain of claim 1.

31. A method of producing the bioniat of claim 30 comprising
(a) inoculating an artificial liquid growth medium with an effective amount of the recombinant filamentous fungal strain;
(b) incubating the inoculated filamentous fungi to produce a filamentous fungal biomat on the surface of the liquid medium; and
(c) harvesting the filamentous fungal biomat.

32-34. (canceled)
